SEQUENCE LISTING (164 sequences)

SEQ ID NO: 1 - protein, 23 aa, Homo sapiens
YKCMECGKAFNRRSHLTRHQRIH

SEQ ID NO: 2 - protein, 23 aa, Homo sapiens
FKCPVCGKAFRHSSSLVRHQRTH

SEQ ID NO: 3 - protein, 24 aa, Homo sapiens
YRCKYCDRSFSISSNLQRHVRNIH

SEQ ID NO: 4 - protein, 23 aa, Homo sapiens
YTCSYCGKSFTQSNTLKQHTRIH

SEQ ID NO: 5 - protein, 23 aa, Homo sapiens
YKCKQCGKAFGCPSNLRRHGRTH

SEQ ID NO: 6 - protein, 24 aa, Homo sapiens
YRCKYCDRSFSISSNLQRHVRNIH

SEQ ID NO: 7 - protein, 24 aa, Homo sapiens
YRCKYCDRSFSISSNLQRHVRNIH

SEQ ID NO: 8 - protein, 23 aa, Homo sapiens
FQCKTCQRKFSRSDHLKTHTRTH

SEQ ID NO: 9 - protein, 23 aa, Homo sapiens
YECHDCGKSFRQSTHLTRHRRIH

SEQ ID NO: 10 - protein, 23 aa, Homo sapiens
YECNYCGKTFSVSSTLIRHQRIH

SEQ ID NO: 11 - protein, 23 aa, Homo sapiens
YECDHCGKSFSQSSHLNVHKRTH

SEQ ID NO: 12 - protein, 23 aa, Homo sapiens
YECDHCGKAFSVSSNLNVHRRIH

SEQ ID NO: 13 - protein, 23 aa, Homo sapiens
YKCEECGKAFTQSSNLTKHKKIH

SEQ ID NO: 14 - protein, 23 aa, Homo sapiens
YKCEECGKAFTQSSNLTKHKKIH

SEQ ID NO: 15 - protein, 23 aa, Homo sapiens
FQCKTCQRKFSRSDHLKTHTRTH

SEQ ID NO: 16 - protein, 23 aa, Homo sapiens
YECDHCGKSFSQSSHLNVHKRTH

SEQ ID NO: 17 - protein, 23 aa, Homo sapiens
YECHDCGKSFRQSTHLTRHRRIH

SEQ ID NO: 18 - protein, 23 aa, Homo sapiens
YKCPDCGKSFSQSSSLIRHQRTH

SEQ ID NO: 19 - protein, 23 aa, Homo sapiens
YRCEECGKAFRWPSNLTRHKRIH

SEQ ID NO: 20 - protein, 23 aa, Homo sapiens
YECDHCGKSFSQSSHLNVHKRTH

SEQ ID NO: 21 - protein, 23 aa, Homo sapiens
YECDHCGKAFSVSSNLNVHRRIH

SEQ ID NO: 22 - protein, 23 aa, Homo sapiens
YECDHCGKSFSQSSHLNVHKRTH

SEQ ID NO: 23 - protein, 23 aa, Homo sapiens
YECHDCGKSFRQSTHLTRHRRIH

SEQ ID NO: 24 - protein, 23 aa, Homo sapiens
YKCMECGKAFNRRSHLTRHQRIH

SEQ ID NO: 25 - protein, 23 aa, Homo sapiens
YECHDCGKSFRQSTHLTRHRRIH

SEQ ID NO: 26 - protein, 23 aa, Homo sapiens
YECHDCGKSFRQSTHLTRHRRIH

SEQ ID NO: 27 - protein, 23 aa, Homo sapiens
FMCTWSYCGKRFTDRSARHKRTH

SEQ ID NO: 28 - protein, 23 aa, Homo sapiens
FQCKTCQRKFSRSDHLKTHTRTH

SEQ ID NO: 29 - protein, 23 aa, Homo sapiens
YECDHCGKSFSQSSHLNVHKRTH

SEQ ID NO: 30 - protein, 23 aa, Homo sapiens
YECHDCGKSFRQSTHLTRHRRIH

SEQ ID NO: 31 - protein, 23 aa, Homo sapiens
YECHDCGKSFRQSTHLTQHRRIH

SEQ ID NO: 32 - protein, 23 aa, Homo sapiens
YKCMECGKAFNRRSHLTRHQRIH

SEQ ID NO: 33 - protein, 23 aa, Homo sapiens
YECHDCGKSFRQSTHLTRHRRIH

SEQ ID NO: 34 - protein, 23 aa, Homo sapiens
YECHDCGKSFRQSTHLTRHRRIH

SEQ ID NO: 35 - protein, 23 aa, Homo sapiens
YECDHCGKSFSQSSHLNVHKRTH

SEQ ID NO: 36 - protein, 23 aa, Homo sapiens
YECDHCGKAFSVSSNLNVHRRIH

SEQ ID NO: 37 - protein, 23 aa, Homo sapiens
FECKDCGKAFIQKSNLIRHQRTH

SEQ ID NO: 38 - protein, 23 aa, Homo sapiens
YKCKQCGKAFGCPSNLRRHGRTH

SEQ ID NO: 39 - protein, 23 aa, Homo sapiens
YECDHCGKAFSVSSNLNVHRRIH

SEQ ID NO: 40 - protein, 23 aa, Homo sapiens
YECHDCGKSFRQSTHLTRHRRIH

SEQ ID NO: 41 - protein, 23 aa, Homo sapiens
YKCPDCGKSFSQSSSLIRHQRTH

SEQ ID NO: 42 - protein, 23 aa, Homo sapiens
FQCKTCQRKFSRSDHLKTHTRTH

SEQ ID NO: 43 - protein, 23 aa, Homo sapiens
FQCKTCQRKFSRSDHLKTHTRTH

SEQ ID NO: 44 - protein, 23 aa, Homo sapiens
YECDHCGKSFSQSSHLNVHKRTH

SEQ ID NO: 45 - protein, 23 aa, Homo sapiens
YECHDCGKSFRQSTHLTRHRRIH

SEQ ID NO: 46 - protein, 23 aa, Homo sapiens
FECKDCGKAFIQKSNLIRHQRTH

SEQ ID NO: 47 - protein, 23 aa, Homo sapiens
FMCTWSYCGKRFTDRSARHKRTH

SEQ ID NO: 48 - protein, 23 aa, Homo sapiens
FQCKTCQRKFSRSDHLKTHTRTH

SEQ ID NO: 49 - protein, 23 aa, Homo sapiens
YKCEECGKAFTQSSNLTKHKKIH

SEQ ID NO: 50 - protein, 23 aa, Homo sapiens
YECHDCGKSFRQSTHLTRHRRIH

SEQ ID NO: 51 - protein, 80 aa, Artificial Sequence
  Synthetically generated peptide
YKCMECGKAFNRRSHLTRHQRIHTGEKPFKCPVCGKAFRHSSSLVRHQRTHTGEKPYRCKYCDRSFSISSNLQRHVRNIH

SEQ ID NO: 52 - protein, 80 aa, Artificial Sequence
  Synthetically generated peptide
YTCSYCGKSFTQSNTLKQHTRIHTGEKPYKCKQCGKAFGCPSNLRRHGRTHTGEKPYRCKYCDRSFSISSNLQRHVRNIH

SEQ ID NO: 53 - protein, 108 aa, Artificial Sequence
  Synthetically generated peptide
YRCKYCDRSFSISSNLQRHVRNIHTGEKPFQCKTCQRKFSRSDHLKTHTRTHTGEKPYECHDCGKSFRQSTHLTRHRRIHTGEKPYECNYCGKTFSVSSTLIRHQRIH

SEQ ID NO: 54 - protein, 107 aa, Artificial Sequence
  Synthetically generated peptide
YECDHCGKSFSQSSHLNVHKRTHTGEKPYECDHCGKAFSVSSNLNVHRRIHTGEKPYKCEECGKAFTQSSNLTKHKKIHTGEKPYKCEECGKAFTQSSNLTKHKKIH

SEQ ID NO: 55 - protein, 107 aa, Artificial Sequence
  Synthetically generated peptide
FQCKTCQRKFSRSDHLKTHTRTHTGEKPYECDHCGKSFSQSSHLNVHKRTHTGEKPYECHDCGKSFRQSTHLTRHRRIHTGEKPYKCPDCGKSFSQSSSLIRHQRTH

SEQ ID NO: 56 - protein, 107 aa, Artificial Sequence
  Synthetically generated peptide
YRCEECGKAFRWPSNLTRHKRIHTGEKPYECDHCGKSFSQSSHLNVHKRTHTGEKPYECDHCGKAFSVSSNLNVHRRIHTGEKPYECDHCGKSFSQSSHLNVHKRTH

SEQ ID NO: 57 - protein, 107 aa, Artificial Sequence
  Synthetically generated peptide
YECHDCGKSFRQSTHLTRHRRIHTGEKPYKCMECGKAFNRRSHLTRHQRIHTGEKPYECHDCGKSFRQSTHLTRHRRIHTGEKPYECHDCGKSFRQSTHLTRHRRIH

SEQ ID NO: 58 - protein, 107 aa, Artificial Sequence
  Synthetically generated peptide
FMCTWSYCGKRFTDRSARHKRTHTGEKPFQCKTCQRKFSRSDHLKTHTRTHTGEKPYECDHCGKSFSQSSHLNVHKRTHTGEKPYECHDCGKSFRQSTHLTRHRRIH

SEQ ID NO: 59 - protein, 107 aa, Artificial Sequence
  Synthetically generated peptide
YECHDCGKSFRQSTHLTQHRRIHTGEKPYKCMECGKAFNRRSHLTRHQRIHTGEKPYECHDCGKSFRQSTHLTRHRRIHTGEKPYECHDCGKSFRQSTHLTRHRRIH

SEQ ID NO: 60 - protein, 107 aa, Artificial Sequence
  Synthetically generated peptide
YECDHCGKSFSQSSHLNVHKRTHTGEKPYECDHCGKAFSVSSNLNVHRRIHTGEKPFECKDCGKAFIQKSNLIRHQRTHTGEKPYKCKQCGKAFGCPSNLRRHGRTH

SEQ ID NO: 61 - protein, 107 aa, Artificial Sequence
  Synthetically generated peptide
YECDHCGKAFSVSSNLNVHRRIHTGEKPYECHDCGKSFRQSTHLTRHRRIHTGEKPYKCPDCGKSFSQSSSLIRHQRTHTGEKPFQCKTCQRKFSRSDHLKTHTRTH

SEQ ID NO: 62 - protein, 107 aa, Artificial Sequence
  Synthetically generated peptide
FQCKTCQRKFSRSDHLKTHTRTHTGEKPYECDHCGKSFSQSSHLNVHKRTHTGEKPYECHDCGKSFRQSTHLTRHRRIHTGEKPFECKDCGKAFIQKSNLIRHQRTH

SEQ ID NO: 63 - protein, 107 aa, Artificial Sequence
  Synthetically generated peptide
FMCTWSYCGKRFTDRSARHKRTHTGEKPFQCKTCQRKFSRSDHLKTHTRTHTGEKPYKCEECGKAFTQSSNLTKHKKIHTGEKPYECHDCGKSFRQSTHLTRHRRIH

SEQ ID NO: 64 - protein, 13 aa, Simian parainfluenza virus 5
GKPIPNPLLGLDS

SEQ ID NO: 65 - protein, 89 aa, Artificial Sequence
  Synthetically generated peptide
ERPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 66 - protein, 21 aa, Artificial Sequence
  Synthetically generated peptide
XXCXXXXXCXXXXXHXXXXXH

SEQ ID NO: 67 - protein, 23 aa, Homo sapiens
YKCKQCGKAFGCPSNLRRHGRTH

SEQ ID NO: 68 - protein, 23 aa, Homo sapiens
YQCNICGKCFSCNSNLHRHQRTH

SEQ ID NO: 69 - protein, 23 aa, Homo sapiens
YSCGICGKSFSDSSAKRRHCILH

SEQ ID NO: 70 - protein, 23 aa, Homo sapiens
YTCSDCGKAFRDKSCLNRHRRTH

SEQ ID NO: 71 - protein, 23 aa, Homo sapiens
YKCKECGKAFNHSSNFNKHHRIH

SEQ ID NO: 72 - protein, 23 aa, Homo sapiens
FKCPVCGKAFRHSSSLVRHQRTH

SEQ ID NO: 73 - protein, 24 aa, Homo sapiens
YRCKYCDRSFSISSNLQRHVRNIH

SEQ ID NO: 74 - protein, 23 aa, Homo sapiens
YECDHCGKAFSIGSNLNVHRRIH

SEQ ID NO: 75 - protein, 23 aa, Homo sapiens
YGCHLCGKAFSKSSNLRRHEMIH

SEQ ID NO: 76 - protein, 23 aa, Homo sapiens
YKCKECGQAFRQRAHLIRHHKLH

SEQ ID NO: 77 - protein, 23 aa, Homo sapiens
YKCHQCGKAFIQSFNLRRHERTH

SEQ ID NO: 78 - protein, 23 aa, Homo sapiens
FQCNQCGASFTQKGNLLRHIKLH

SEQ ID NO: 79 - protein, 23 aa, Homo sapiens
YACHLCGKAFTQSSHLRRHEKTH

SEQ ID NO: 80 - protein, 23 aa, Homo sapiens
YKCGQCGKFYSQVSHLTRHQKIH

SEQ ID NO: 81 - protein, 23 aa, Homo sapiens
YACHLCGKAFTQCSHLRRHEKTH

SEQ ID NO: 82 - protein, 23 aa, Homo sapiens
YACHLCAKAFIQCSHLRRHEKTH

SEQ ID NO: 83 - protein, 23 aa, Homo sapiens
YVCRECGRGFRQHSHLVRHKRTH

SEQ ID NO: 84 - protein, 23 aa, Homo sapiens
YKCEECGKAFRQSSHLTTHKIIH

SEQ ID NO: 85 - protein, 23 aa, Homo sapiens
YECDHCGKSFSQSSHLNVHKRTH

SEQ ID NO: 86 - protein, 23 aa, Homo sapiens
YMCSECGRGFSQKSNLIIHQRTH

SEQ ID NO: 87 - protein, 23 aa, Homo sapiens
YKCEECGKAFTQSSNLTKHKKIH

SEQ ID NO: 88 - protein, 23 aa, Homo sapiens
FECKDCGKAFIQKSNLIRHQRTH

SEQ ID NO: 89 - protein, 23 aa, Homo sapiens
YVCRECRRGFSQKSNLIRHQRTH

SEQ ID NO: 90 - protein, 23 aa, Homo sapiens
YECEKCGKAFNQSSNLTRHKKSH

SEQ ID NO: 91 - protein, 23 aa, Homo sapiens
YECNTCRKTFSQKSNLIVHQRTH

SEQ ID NO: 92 - protein, 23 aa, Homo sapiens
YVCSKCGKAFTQSSNLTVHQKIH

SEQ ID NO: 93 - protein, 23 aa, Homo sapiens
YKCDECGKNFTQSSNLIVHKRIH

SEQ ID NO: 94 - protein, 23 aa, Homo sapiens
YECDVCGKTFTQKSNLGVHQRTH

SEQ ID NO: 95 - protein, 23 aa, Homo sapiens
YECVQCGKGFTQSSNLITHQRVH

SEQ ID NO: 96 - protein, 23 aa, Homo sapiens
YKCPDCGKSFSQSSSLIRHQRTH

SEQ ID NO: 97 - protein, 23 aa, Homo sapiens
YECQDCGRAFNQNSSLGRHKRTH

SEQ ID NO: 98 - protein, 23 aa, Homo sapiens
YECNECGKFFSQSSSLIRHRRSH

SEQ ID NO: 99 - protein, 23 aa, Homo sapiens
YKCEECGKAFNQSSTLTRHKIVH

SEQ ID NO: 100 - protein, 23 aa, Homo sapiens
YECNECGKAFAQNSTLRVHQRIH

SEQ ID NO: 101 - protein, 23 aa, Homo sapiens
YECHDCGKSFRQSTHLTQHRRIH

SEQ ID NO: 102 - protein, 23 aa, Homo sapiens
YECHDCGKSFRQSTHLTRHRRIH

SEQ ID NO: 103 - protein, 22 aa, Homo sapiens
HKCLECGKCFSQNTHLTRHQRT

SEQ ID NO: 104 - protein, 25 aa, Homo sapiens
YVCDVEGCTWKFARSDELNRHKKRH

SEQ ID NO: 105 - protein, 25 aa, Homo sapiens
YHCDWDGCGWKFARSDELTRHYRKH

SEQ ID NO: 106 - protein, 25 aa, Homo sapiens
YRCSWEGCEWRFARSDELTRHFRKH

SEQ ID NO: 107 - protein, 25 aa, Homo sapiens
FSCSWKGCERRFARSDELSRHRRTH

SEQ ID NO: 108 - protein, 25 aa, Homo sapiens
FACSWQDCNKKFARSDELARHYRTH

SEQ ID NO: 109 - protein, 25 aa, Homo sapiens
YHCNWDGCGWKFARSDELTRHYRKH

SEQ ID NO: 110 - protein, 24 aa, Homo sapiens
FLCQYCAQRFGRKDHLTRHMKKSH

SEQ ID NO: 111 - protein, 23 aa, Homo sapiens
FQCKTCQRKFSRSDHLKTHTRTH

SEQ ID NO: 112 - protein, 23 aa, Homo sapiens
FACEVCGVRFTRNDKLKIHMRKH

SEQ ID NO: 113 - protein, 25 aa, Homo sapiens
YVCDVEGCTWKFARSDKLNRHKKRH

SEQ ID NO: 114 - protein, 23 aa, Homo sapiens
YKCMECGKAFNRRSHLTRHQRIH

SEQ ID NO: 115 - protein, 23 aa, Homo sapiens
YICRKCGRGFSRKSNLIRHQRTH

SEQ ID NO: 116 - protein, 23 aa, Homo sapiens
YLCSECDKCFSRSTNLIRHRRTH

SEQ ID NO: 117 - protein, 23 aa, Homo sapiens
YECKECGKAFSSGSNFTRHQRIH

SEQ ID NO: 118 - protein, 23 aa, Homo sapiens
YECDHCGKAFSVSSNLNVHRRIH

SEQ ID NO: 119 - protein, 23 aa, Homo sapiens
YTCKQCGKAFSVSSSLRRHETTH

SEQ ID NO: 120 - protein, 23 aa, Homo sapiens
YECNYCGKTFSVSSTLIRHQRIH

SEQ ID NO: 121 - protein, 23 aa, Homo sapiens
YRCEECGKAFRWPSNLTRHKRIH

SEQ ID NO: 122 - protein, 6 aa, Artificial Sequence
  Naturally occurring linker peptide
TGXXPX

SEQ ID NO: 123 - protein, 26 aa, Artificial Sequence
  Synthetically generated peptide
XXCXXXXXCXXXCXSNXXRHXXXXXH

SEQ ID NO: 124 - DNA, 267 bp, Artificial Sequence
  Synthetically generated oligonucleotide
atcgataagctaattctcactcattaggcaccccaggctttacactttatgcttccggctcgtataatgtgtggaattgtgagcggataacaatttcacacaggaaacagcgtccatgggtaagcctatccctaaccctctcctcggtctcgattctacacaagctatgggtgctcctccaaaaaagaagagaaaggtagctggatccactagtaacggccgccagtgtgctggaattctgcagatatccatcacactggcggccgc

SEQ ID NO: 125 - protein, 25 aa, Artificial Sequence
  mutated sequence
FMCTWSYCGKRFTDRSALARHKRTH

SEQ ID NO: 126 - protein, 23 aa, Homo sapiens
YKCKQCGKAFGCPSNLRRHGRTH

SEQ ID NO: 127 - protein, 23 aa, Homo sapiens
YTCSDCGKAFRDKSCLNRHRRTH

SEQ ID NO: 128 - protein, 25 aa, Artificial Sequence
  mutated sequence
YACPVESCDRRFSDSSNLTRHIRIH

SEQ ID NO: 129 - protein, 23 aa, Homo sapiens
FKCPVCGKAFRHSSSLVRHQRTH

SEQ ID NO: 130 - protein, 24 aa, Homo sapiens
YRCKYCDRSFSISSNLQRHVRNIH

SEQ ID NO: 131 - protein, 23 aa, Homo sapiens
YKCHQCGKAFIQSFNLRRHERTH

SEQ ID NO: 132 - protein, 23 aa, Drosophila
YTCSYCGKSFTQSNTLKQHTRIH

SEQ ID NO: 133 - protein, 23 aa, Homo sapiens
YECDHCGKSFSQSSHLNVHKRTH

SEQ ID NO: 134 - protein, 23 aa, Homo sapiens
YMCSECGRGFSQKSNLIIHQRTH

SEQ ID NO: 135 - protein, 23 aa, Homo sapiens
YKCEECGKAFTQSSNLTKHKKIH

SEQ ID NO: 136 - protein, 23 aa, Homo sapiens
FECKDCGKAFIQKSNLIRHQRTH

SEQ ID NO: 137 - protein, 23 aa, Homo sapiens
YVCSKCGKAFTQSSNLTVHQKIH

SEQ ID NO: 138 - protein, 23 aa, Homo sapiens
YKCPDCGKSFSQSSSLIRHQRTH

SEQ ID NO: 139 - protein, 23 aa, Homo sapiens
YECHDCGKSFRQSTHLTQHRRIH

SEQ ID NO: 140 - protein, 23 aa, Homo sapiens
YECHDCGKSFRQSTHLTRHRRIH

SEQ ID NO: 141 - protein, 23 aa, Homo sapiens
FQCKTCQRKFSRSDHLKTHTRTH

SEQ ID NO: 142 - protein, 25 aa, Homo sapiens
YVCDVEGCTWKFARSDKLNRHKKRH

SEQ ID NO: 143 - protein, 23 aa, Artificial Sequence
  mutated sequence
FACPECPKRFMRSDNLTQHIKTH

SEQ ID NO: 144 - protein, 23 aa, Homo sapiens
YKCMECGKAFNRRSHLTRHQRIH

SEQ ID NO: 145 - protein, 23 aa, Homo sapiens
YICRKCGRGFSRKSNLIRHQRTH

SEQ ID NO: 146 - protein, 23 aa, Homo sapiens
YECDHCGKAFSVSSNLNVHRRIH

SEQ ID NO: 147 - protein, 23 aa, Homo sapiens
YTCKQCGKAFSVSSSLRRHETTH

SEQ ID NO: 148 - protein, 23 aa, Homo sapiens
YECNYCGKTFSVSSTLIRHQRIH

SEQ ID NO: 149 - protein, 23 aa, Homo sapiens
YRCEECGKAFRWPSNLTRHKRIH

SEQ ID NO: 150 - DNA, 12 bp, Artificial Sequence
  putative target sequence
daadaaaathga

SEQ ID NO: 151 - DNA, 13 bp, Artificial Sequence
  putative target sequence
gyagrahganggk

SEQ ID NO: 152 - DNA, 12 bp, Artificial Sequence
  putative target sequence
hgaaathgaggt

SEQ ID NO: 153 - DNA, 12 bp, Artificial Sequence
  putative target sequence
gragraggggra

SEQ ID NO: 154 - DNA, 12 bp, Artificial Sequence
  putative target sequence
grahgangggtc

SEQ ID NO: 155 - DNA, 12 bp, Artificial Sequence
  putative target sequence
gragraggghga

SEQ ID NO: 156 - DNA, 12 bp, Artificial Sequence
  putative target sequence
gavgaaaathga

SEQ ID NO: 157 - DNA, 12 bp, Artificial Sequence
  putative target sequence
ngggyagraaat

SEQ ID NO: 158 - DNA, 13 bp, Artificial Sequence
  putative target sequence
gaagrahganggk

SEQ ID NO: 159 - DNA, 12 bp, Artificial Sequence
  putative target sequence
gradaangggtc

SEQ ID NO: 160 - DNA, 12 bp, Artificial Sequence
  binding sequence
gaagrahgangg

SEQ ID NO: 161 - protein, 189 aa, Escherichia coli
MKRLIVGISGASGAIYGVRLLQVLRDVTDIETHLVMSQAARQTLSLETDFSLREVQALADVTHDARDIAASISSGSFQTLGMVILPCSIKTLSGIVHSYTDGLLTRAADVVLKERRPLVLCVRETPLHLGHLRLMTQAAEIGAVIMPPVPAFYHRPQSLDDVINQTVNRVLDQFAITLPEDLFARWQGA

SEQ ID NO: 162 - DNA, 25 bp, Artificial Sequence
  primer
ctggaaagaaccggaagagatgctg

SEQ ID NO: 163 - DNA, 25 bp, Artificial Sequence
  primer
tgaaacgactcattgtaggcatcag

SEQ ID NO: 164 - DNA, 12 bp, Artificial Sequence
  target sequence
gctgrangggah

(19) United States
(12) Patent Application Publication
(10) Pub. No.: US 2004/0259258 A1
(43) Pub. Date: Dec. 23, 2004

(54) Regulation of prokaryotic gene expression with zinc finger proteins

(76) Inventors: Jin-Soo Kim, Yuseong-gu (KR); Kyung-Soon Park, Yuseong-gu (KR); Young-Soon Jang, Yuseong-gu (KR)

Correspondence Address: FISH & RICHARDSON PC, 225 FRANKLIN ST, BOSTON, MA, 02110, US

(21) Appl. No.: 10/746,864

(22) Filed: Dec. 23, 2003

Related U.S. Application Data
(63) Continuation-in-part of application No. 10/669,861, filed on Sep. 24, 2003.
Application No. 10/669,861 is a continuation-in-part of application No. 10/314,669, filed on Dec. 9, 2002.
(60) Provisional application No. 60/401,089, filed on Aug. 5, 2002.
(60) Provisional application No. 60/400,904, filed on Aug. 2, 2002.
(60) Provisional application No. 60/376,053, filed on Apr. 26, 2002.
(60) Provisional application No. 60/338,441, filed on Dec. 7, 2001.

(51) Int. Cl. C12N 1/20; C12N 15/74

(52) U.S. Cl. 435/488; 435/252.33

(57) ABSTRACT

Chimeric zinc finger proteins, and methods of using zinc finger proteins for regulating gene expression in prokaryotes are disclosed herein.

### BACKGROUND

Most genes are regulated at the transcriptional level by polypeptide transcription factors that bind to specific DNA sites within the gene, typically in promoter or enhancer regions. These proteins activate or repress transcriptional initiation by RNA polymerase at the promoter, thereby regulating expression of the target gene. Many transcription factors, both activators and repressors, include structurally distinct domains that have specific functions, such as DNA binding, dimerization, or interaction with the transcriptional machinery. The DNA binding portion of the transcription factor itself can be composed of independent structural domains that contact DNA. The three-dimensional structures of many DNA-binding domains, including zinc finger domains, homeodomains, and helix-turn-helix domains, have been determined from NMR and X-ray crystallographic data. Effector domains such as activation domains or repression domains retain their function when transferred to DNA-binding domains of heterologous transcription factors (Brent and Ptashne, (1985) Cell 43:729-36; Dawson et al., (1995) Mol. Cell Biol. 15:6923-31).

Artificial transcription factors can be produced that are chimeras of zinc finger domains. For example, WO 01/60970 (Kim et al.) describes methods for determining the specificity of zinc finger domains and for constructing artificial transcription factors that recognize particular target sites.

In bacteria, genes are grouped into operons, which are gene clusters that encode the proteins necessary to perform coordinated function, such as biosynthesis of a given amino acid. RNA that is transcribed from a prokaryotic operons is polycistronic, such that multiple proteins are encoded in a single transcript. Gene expression in bacteria can be controlled at the level of transcription initiation, which is regulated by DNA sequence elements upstream of the site of transcriptional initiation that are recognized and contacted by RNA polymerase. RNA polymerase can be regulated, in turn, by interaction with accessory proteins, which can act both positively (activators) and negatively (repressors). The mechanisms by which transcription is regulated in prokaryotes are thought to be less complex than those observed in eukaryotic organisms.

### SUMMARY

The invention provides methods and compositions for regulating gene expression in prokaryotes. In one aspect, the invention features a method of regulating expression of a gene in a prokaryotic cell, the method including: providing a prokaryotic cell comprising a nucleic acid encoding an polypeptide (e.g., an artificial, chimeric polypeptide), wherein the polypeptide comprises a zinc finger domain, and wherein the polypeptide binds to a target DNA site in a gene; expressing the nucleic acid encoding the polypeptide in the cell under conditions in which the polypeptide is produced, binds to the target DNA site, and regulates the gene.

The artificial polypeptide can include two, three, four, five, six, or more zinc finger domains. In one embodiment, the artificial polypeptide includes three zinc finger domains. In one embodiment, the artificial polypeptide includes four zinc finger domains. In one embodiment, the artificial polypeptide includes five or more zinc finger domains.

The zinc finger domain or domains of the artificial polypeptide can be naturally-occurring zinc finger domains or variants thereof. In one embodiment, each zinc finger domain of the artificial polypeptide is identical to a naturally-occurring zinc finger domain. In one embodiment, the artificial polypeptide includes a first zinc finger domain that is identical to a naturally-occurring zinc finger domain, and a second zinc finger domain that is a variant of a naturally-occurring zinc finger domain.

In one embodiment, the artificial polypeptide includes two zinc finger domains, wherein each of the two zinc finger domains is identical to a zinc finger domain of a same naturally-occurring protein, or a variant thereof. In one embodiment, the artificial polypeptide includes two zinc finger domains, wherein the each of the zinc finger domains is identical to a zinc finger domain of a different naturally-occurring protein, or a variant thereof. In one embodiment, the artificial polypeptide includes two zinc finger domains, and each of the two zinc finger domains is identical to a non-adjacent zinc finger domain of a same naturally-occurring protein.

The artificial polypeptide can include one or more of the following features:

the artificial polypeptide regulates expression of an endogenous gene; the artificial polypeptide regulates expression of an exogenous (e.g., heterologous) gene; the artificial polypeptide regulates expression of a phage gene; the artificial polypeptide regulates expression of a transposon gene; the artificial polypeptide has a dissociation constant for a DNA site of less than 50 nM; the artificial polypeptide includes one or more zinc finger domains, wherein the DNA contacting residues of one or more of the zinc finger domains at positions −1, +2, +3, and +6 correspond to an amino acid motif selected from the following: RSHR, HSSR, ISNR, RDHT, QTHR, VSTR, QNTQ, CSNR, QSHV, VSNV, QSNK, QSSR, WSNR, DSAR, QTHQ, QSNR, and CSNR. In one embodiment, the non-DNA contacting residues are identical to a set of non-DNA contacting residues described herein. For example, the zinc finger domain can include a zinc finger domain from Table 1.

TABLE 1

| | | |
|---|---|---|
| ZFD | Amino Acid Sequence | SEQ ID NO: |

| | | |
|---|---|---|
| H1.1 | YKCMECGKAFNRRSHLTRHQRIH | 1 |
| H1.2 | FKCPVCGKAFRHSSSLVRHQRTH | 2 |
| H1.3 | YRCKYCDRSFSISSNLQRHVRNIH | 3 |
| H2.1 | YTCSYCGKSF TQSNTLKQHTRIH | 4 |
| H2.2 | YKCKQCGKAFGCPSNLRRHGRTH | 5 |
| H2.3 | YRCKYCDRSFSISSNLQRHVRNIH | 6 |
| H3.1 | YRCKYCDRSFSISSNLQRHVRNIH | 7 |
| H3.2 | FQCKTCQRKFSRSDHLKTHTRTH | 8 |
| H3.3 | YECHDCGKSFRQSTHLTRHRRIH | 9 |
| H3.4 | YECNYCGKTFSVSSTLIRHQRIH | 10 |
| T1.1 | YECDHCGKSFSQSSHLNVHKRTH | 11 |
| T1.2 | YECDHCGKAFSVSSNLNVHRRIH | 12 |
| T1.3 | YKCEECGKAFTQSSNLTKHKKIH | 13 |
| T1.4 | YKCEECGKAFTQSSNL TKHKKIH | 14 |
| T2.1 | FQCKTCQRKFSRSDHLKTHTRTH | 15 |
| T2.2 | YE CDHCGKSFSQSSHLNVHKRTH | 16 |
| T2.3 | YECHDCGKSFRQSTHLTRHRRIH | 17 |
| T2.4 | YKCPDCGKSFSQSSSLIRHQRTH | 18 |
| T3.1 | YRCEECGKAFRWPSNLTRHKRIH | 19 |
| T3.2 | YECDHCGKSFSQSSHLNVHKRTH | 20 |
| T3.3 | YECDHCGKAFSVSSNLNVHRRIH | 21 |
| T3.4 | YECDHCGKSFSQSSHLNVHKRTH | 22 |
| T4.1 | YECHDCGKSFRQSTHLTRHRRIH | 23 |
| T4.2 | YKCMECGKAFNRRSHLTRHQRIH | 24 |
| T4.3 | YECHDCGKSFRQSTHLTRHRRIH | 25 |
| T4.4 | YECHDCGKSFRQSTHLTRHRRIH | 26 |
| T5.1 | FMCTWSYCGKRFTDRSALARHKRTH | 27 |
| T5.2 | FQCKTCQRKFSRSDHLKTHTRTH | 28 |
| T5.3 | YECDHCGKSFSQSSHLNVHKRTH | 29 |
| T5.4 | YECH DCGKSFRQSTHLTRHRRIH | 30 |
| T6.1 | YECHDCGKSFRQSTHLTQHRRIH | 31 |
| T6.2 | YKCMECGKAFNRRSHLTRHQRIH | 32 |
| T6.3 | YECHDCGKSFRQSTHLTRHRRIH | 33 |
| T6.4 | YECHDC GKSFRQSTHL TRHRRIH | 34 |
| T7.1 | YECDHCGKSFSQSSHLNVHKRTH | 35 |
| T7.2 | YECDHCGKAFSVSSNLNVHRRIH | 36 |
| T7.3 | FECKDCGKAFIQKSNLRHQRTH | 37 |
| T7.4 | YKCKQCGKAFGCPSNLRRHGRTH | 38 |
| T8.1 | YECDHCGKAFSVSSNLNVHRRIH | 39 |
| T8.2 | YECHDCGKSFRQSTHLTRHRRIH | 40 |
| T8.3 | YKCPDCGKSFSQSSSLIRHQRTH | 41 |
| T8.4 | FQCKTCQRKFSRSDHL KTHTRTH | 42 |
| T9.1 | FQCKTCQRKFSRSDHLKTHTRTH | 43 |
| T9.2 | YECDHCGKSFSQSSHLNVHKRTH | 44 |
| T9.3 | YECHDCGKSFRQSTHLTRHRRIH | 45 |
| T9.4 | FECKDCGKAFIQKSNLIRHQRTH | 46 |
| T10.1 | FMCTWSYCGKRFTDRSALARHKRTH | 47 |
| T10.2 | FQCKTCQRKFSRSDHLKTHTRTH | 48 |
| T10.3 | YKCEECGKAFTQSSNLTKHKKIH | 49 |
| T10.4 | YECHDCGKSFRQSTHLTRHRRIH | 50 |

The artificial polypeptide can include an amino acid sequence that differs by 1 to 8 amino acid substitutions, deletions, or insertions from a sequence in Table 1. The substitution may be at a position other than a DNA contacting residue, e.g., between a metal-coordinating cysteine and position −1. The substitutions can be conservative substitutions.

In one embodiment, the artificial polypeptide includes one or more of the zinc finger domains shown in Table 1.

In one embodiment, the artificial polypeptide includes an amino acid sequence at least 75%, 80%, 85%, 90%, 95%, 99%, or 100% identical to a sequence of a zinc finger protein in Table 2.

TABLE 2

| | | |
|---|---|---|
| ZFP | Amino acid Sequence | SEQ ID NO: |

| | | |
|---|---|---|
| H1 | YKCMECGKAFNRRSHLTRHQRIHTGEKPFKCPVCGKAFRHSSSLVRHQRT | 51 |
| | HTGEKPYRCKYCDRSFSISSNLQRHVRNIH | |
| H2 | YTCSYCGKSFTQSNTLKQHTRIHTGEKPYKCKQCGKAFGCPSNLRRHGRT | 52 |
| | HTGEKPYRCKYCDRSFSISSNLQRHVRNIH | |
| H3 | YRCKYCDRSFSISSNLQRHVRNIHTGEKPFQCKTCQRKFSRSDHLKTHTRT | 53 |
| | HTGEKPYECHDCGKSFRQSTHLTRHRRIHTGEKPYECNYCGKTFSVSSTLI | |
| | RHQRIH | |
| T1 | YECDHCGKSFSQSSHLNVHKRTHTGEKPYECDHCGKAFSVSSNLNVHRRI | 54 |
| | HTGEKPYKCEECGKAFTQSSNLTKHKKIHTGEKPYKCEECGKAFTQSSNL | |
| | TKHKKIH | |
| T2 | FQCKTCQRKFSRSDHLKTHTRTHTGEKPYECDHCGKSFSQSSHLNVHKRT | 55 |
| | HTGEKPYECHDCGKSFRQSTHLTRHRRIHTGEKPYKCPDCGKSFSQSSSLI | |
| | RHQRTH | |
| T3 | YRCEECGKAFRWPSNLTRHKRIHTGEKPYECDHCGKSFSQSSHLNVHKRT | 56 |
| | HTGEKPYECDHCGKAFSVSSNLNVHRRIHTGEKPYECDHCGKSFSQSSHL | |
| | NVHKRTH | |
| T4 | YECHDCGKSFRQSTHLTRHRRIHTGEKPYKCMECGKAFNRRSHLTRHQRI | 57 |
| | HTGEKPYECHDCGKSFRQSTHLTRHRRIHTGEKPYECHDCGKSFRQSTHL | |
| | TRHRRIH | |
| T5 | FMCTWSYCGKRFTDRSALARHKRTHTGEKPFQCKTCQRKFSRSDHLKTH | 58 |
| | TRTHTGEKPYECDHCGKSFSQSSHLNVHKRTHTGEKPYECHDCGKSFRQS | |
| | THLTRHRRIH | |
| T6 | YECHDCGKSFRQSTHLTQHRRIHTGEKPYKCMECGKAFNRRSHLTRHQRI | 59 |
| | HTGEKPYECHDCGKSFRQSTHLTRHRRIHTGEKPYECHDCGKSFRQSTHL | |
| | TRHRRIH | |
| T7 | YECDHCGKSFSQSSHLNVHKRTHTGEKPYECDHCGKAFSVSSNLNVHRRI | 60 |
| | HTGEKPFECKDCGKAFIQKSNLIRHQRTHTGEKPYKCKQC GKAFGCPSNL | |
| | RRHGRTH | |
| T8 | YECDHCGKAFSVSSNLNVHRRIHTGEKPYECHDCGKSFRQSTHLTRHRRI | 61 |
| | HTGEKPYKCPDCGKSFSQSSSLIRHQRTHTGEKPFQCKTCQRKFSRSDHL | |
| | KTHTRTH | |
| T9 | FQCKTCQRKFSRSDHLKTHTRTHTGEKPYECDHCGKSFSQSSHLNVHKRT | 62 |
| | HTGEKPYECHDCGKSFRQSTHLTRHRRIHTGEKPFECKDCGKAFIQKSNL | |
| | IRHQRTH | |
| T10 | FMCTWSYCGKRFTDRSALARHKRTHTGEKPFQCKTCQRKFSRSDHLKTH | 63 |
| | TRTHTGEKPYKCEECGKAFTQSSNLTKHKKIHTGEKPYECHDCGKSFRQS | |
| | T HLTRHRRIH | |

The artificial polypeptide can include an epitope tag, e.g., a V5 epitope tag (e.g., having the following amino acid sequence: GKPIPNPLLGLDS (SEQ ID NO:64)).

In one embodiment, the artificial polypeptide binds within 50, 40, 30, 20, or 10 nucleotides of a −35 or −10 element of a prokaryotic gene. In one embodiment, the artificial polypeptide binds a transcription factor binding site or binds a site that overlaps a transcription factor binding site.

Expression of the nucleic acid encoding the artificial polypeptide can be regulatable, e.g., by operably linking the sequence encoding the artificial polypeptide to a regulatable promoter. Regulatable promoters include promoters responsive to thermal changes, hormones, metals, metabolites, antibiotics, or chemical agents. In one embodiment, expression of the nucleic acid encoding the artificial polypeptide is regulatable with IPTG (e.g., the sequence encoding the artificial polypeptide is operably linked to a lac promoter).

The artificial polypeptide can include other features described herein.

In one embodiment, the artificial polypeptide regulates expression of an endogenous gene (e.g., directly or indirectly). In one embodiment, the artificial polypeptide regulates expression of two, three, four, or more endogenous genes. In one embodiment, the artificial polypeptide regulates expression of one or more endogenous genes by modulating transcription of a polycistronic RNA.

The method can further include characterizing the endogenous gene. For example, DNA comprising the target DNA site of the artificial polypeptide can be isolated (e.g., by cross-linking the artificial protein to the DNA, immunoprecipitating the artificial protein, and isolating the DNA associated with the protein), and nucleotides associated with the target DNA site can be sequenced. A gene associated with the target DNA site can be identified. The method can further include identifying a homolog of the endogenous gene in a second cell, and regulating the expression of the homolog in the second cell. The second cell can be a prokaryotic cell or a eukaryotic cell.

In one embodiment, the artificial polypeptide regulates expression of a heterologous gene. In one embodiment, the artificial polypeptide regulates expression of two, three, or more heterologous genes.

In one embodiment, the artificial polypeptide includes a transcriptional activation domain. In one embodiment, the artificial polypeptide includes a transcriptional repression domain.

In one embodiment, expression of the gene is repressed (e.g., relative to expression of the gene in the absence of the artificial protein, or relative to a reference value). In one embodiment, expression of the gene is activated (e.g., relative to expression of the gene in the absence of the artificial protein, or relative to a reference value).

In one embodiment, the cell is a bacterial cell, e.g., an E. coli cell. The cell can be any prokaryotic cell, e.g., a Gram-negative bacterial cell, a Gram-positive bacterial cell, a pathogenic bacterial cell, a non-pathogenic bacterial cell (e.g., a commensal bacterial cell). The cell can be selected from a cell of one of the following species: Mycobacterium spp. (e.g., Mycobacterium tuberculosis, Mycobacterium leprae), Lactobacillus spp., Streptococcus spp. (e.g., Streptococcus pneumoniae, Streptococcus pyogenes), Staphylococcus spp. (e.g., Staphylococcus aureus), Bacillus spp. (e.g., Bacillus subtilis, Bacillus anthracis), Campylobacter spp., Pseudomonas spp. (e.g., Pseudomonas aeruginosa), Clostridium spp. (e.g., Clostridium tetani, Clostridium botulinum, Clostridium perfringens), Salmonella spp. (e.g., Salmonella typhi), Corynebacteria spp. (e.g., Corynebacteria diphtheriae), Escherichia spp. (e.g., Escherichia coli), and Listeria spp. (e.g., Listeria monocytogenes), Streptomyces spp., and Thermobifida spp.

A plurality of cells can be provided.

The regulating can alter a trait of the cell relative to a reference cell, e.g., a cell that does not express the artificial polypeptide. The trait can be any detectable phenotype, e.g., a phenotype that can be observed, selected, inferred, and/or quantitated. Traits include: heat resistance, solvent resistance, heavy metal resistance, osmolarity resistance, resistance to extreme pH, chemical resistance, cold resistance, and resistance to a genotoxic agent, resistance to radioactivity.

For example, the trait is resistance to an environmental condition, e.g., heavy metals, salinity, environmental toxins, biological toxins, pathogens, parasites, other environmental extremes (e.g., desiccation, heat, cold), and so forth. In a related example, the trait is stress resistance (e.g., to heat, cold, extreme pH, chemicals, such as ammonia, drugs, osmolarity, and ionizing radiation). In yet another example, the trait is drug resistance. The change in the trait can be in either direction, e.g., towards sensitivity or further resistance.

In one embodiment, the artificial polypeptide regulates expression of an endogenous gene which is a decarboxylase enzyme. In one embodiment, the decarboxylase enzyme is a decarboxylase enzyme of a ubiquinone biosynthetic pathway, e.g., a ubiX gene product of E. coli.

In another aspect, the invention features a method including: providing a plurality of prokaryotic cells, wherein each cell of the plurality comprises a nucleic acid encoding an artificial polypeptide, wherein the artificial polypeptide comprises a zinc finger domain, and wherein the artificial polypeptide differs among the cells of the plurality; and, identifying from the plurality a cell that has a trait that is altered relative to a reference cell. The reference cell can be a cell that does not include a nucleic acid encoding the artificial polypeptide, e.g., the reference cell is a parental cell from which the plurality of cells was made, or a derivative thereof.

The trait can be any detectable phenotype, e.g., a phenotype that can be observed, selected, inferred, and/or quantitated. The artificial polypeptide can be a chimeric polypeptide. As used herein, a chimeric polypeptide includes at least two binding domains that are heterologous to each other (e.g., two zinc finger domains). The two binding domains can be from different naturally occurring proteins. The artificial polypeptide can include one or more features described herein.

In many embodiments, the cell does not include a reporter gene. In other words, the cells can be screened without having, a priori, information about a target gene whose regulation is altered by expression of the chimeric polypeptide. In addition, the cell may include a reporter gene as an additional indicator of a marker that is related or unrelated to the trait. Likewise, one or more target genes may be known prior to the screening.

In another example, the trait is production of a compound (e.g., a natural or artificial compound.

The trait can be resistance to an environmental condition, e.g., heavy metals, salinity, environmental toxins, biological toxins, pathogens, parasites, other environmental extremes (e.g., desiccation, heat, cold), and so forth. In a related example, the trait is stress resistance (e.g., to heat, cold, extreme pH, chemicals, such as ammonia, drugs, osmolarity, and ionizing radiation). In yet another example, the trait is drug resistance. The change in the trait can be in either direction, e.g., towards sensitivity or further resistance.

In one embodiment, the trait is tolerance to an organic solvent, and the identifying comprises exposing cells of the plurality to the organic solvent and evaluating survival of the cells. In one embodiment, the trait is heat tolerance, and the evaluating comprises exposing the cells to heat.

In various embodiments, the identifying includes evaluating cell survival under a set of conditions.

Typically, one or more of the zinc finger domains of the artificial polypeptides varies among nucleic acids of the library. The nucleic acid can also express at least a third DNA binding domain, e.g., a third zinc finger domain.

The cells of the plurality can include nucleic acids encoding a sufficient number of different artificial polypeptides to recognize at least 10, 20 30, 40, or 50 different 3-base pair DNA sites. In one embodiment, the cells of the plurality include nucleic acids encoding a sufficient number of artificial polypeptides to recognize no more than 30, 20, 10, or 5 different 3-base pair DNA sites.

The method can further include isolating the nucleic acid encoding the artificial polypeptide from the identified cell and/or isolating the artificial polypeptide from the identified cell. The nucleic acid encoding the artificial polypeptide can be sequenced.

In one embodiment, the method further includes: isolating the nucleic acid encoding the artificial polypeptide from the cell, introducing the nucleic acid into a second plurality of cells, culturing the cells of the second plurality under conditions wherein the artificial polypeptide is produced, identifying a cell of the second plurality having a trait that is altered relative to a reference cell.

The sequence of the target DNA site of the artificial polypeptide can be determined (e.g., by a computer string or profile search of a sequence database, or by selecting the in vitro nucleic acids that bind to the artificial polypeptide (e.g., SELEX).

The method can further include analyzing the expression of one or more genes of the cell, e.g., using .g., using mRNA profiling (e.g., using microarray analysis), 2-D gel electrophoresis, an array of protein ligands (e.g., antibodies), and/or mass spectroscopy. Also, a single or small number of genes or proteins can also be profiled. In one embodiment, the profile is compared to a database of reference profiles. In another embodiment, regulatory regions of genes whose expression is altered by expression of the identified chimeric polypeptide are compared to identify candidate sites that determine coordinate regulation that results directly or indirectly from expression of the artificial polypeptide.

An endogenous gene bound by the artificial polypeptide can be characterized, e.g., identified by sequencing. Expression of the endogenous gene can be regulated in a second cell, e.g., by a means other than ZFP-mediated regulation, e.g., by knocking out the gene, or overexpressing the gene in the second cell.

The cells of the plurality can include nucleic acids encoding artificial polypeptides comprising naturally-occurring zinc finger domain(s), or variants thereof. The naturally-occurring zinc finger domains can be domains of any eukaryotic zinc finger protein: for example, a fungal (e.g., yeast), plant, or animal protein (e.g., a mammalian protein, such as a human or murine protein).

The cells of the plurality can include nucleic acids encoding artificial polypeptides comprising one, two three, or four zinc finger domains. In one embodiment, the artificial polypeptides include at least three zinc finger domains. The artificial polypeptides encoded by the nucleic acids can include other features described herein.

In one embodiment, the cells of the plurality are E. coli cells.

The method can further include cultivating the identified cell to exploit the altered trait. For example, if the altered trait is increased production of a metabolite, the method can include cultivating the cell to produce the metabolite. The cell can be the cell isolated from the plurality, or a cell into which the nucleic acid encoding the artificial polypeptide has been re-introduced. Expression of the artificial polypeptide can be tuned, e.g., using an inducible promoter, in order to finely vary the trait, or another conditional promoter (e.g., a cell type specific promoter). A cell containing the nucleic acid encoding the artificial polypeptide can be introduced into an organism (e.g., ex vivo treatment).

Exemplary applications of these methods include: identifying essential genes in (e.g., in a pathogenic microbe), identifying genes required for a particular phenotype, identifying targets of drug candidates, gene discovery in signal transduction pathways, microbial engineering and industrial biotechnology, increasing yield of metabolites of commercial interests, and modulating growth behavior (e.g. improving growth of a microorganism).

In another aspect, the invention features a prokaryotic cell including: a nucleic acid encoding an artificial polypeptide, wherein the artificial polypeptide comprises a zinc finger domain, and wherein the artificial polypeptide binds to a target DNA site in a gene and regulates expression of the gene under conditions in which the nucleic acid is expressed. The cell can be an E. coli cell.

In one embodiment, the artificial polypeptide regulates expression of an endogenous gene. In one embodiment, the artificial polypeptide regulates expression of a heterologous gene.

The artificial polypeptide can include one, two, three, four, five, six, or more zinc finger domains. In one embodiment, the artificial polypeptide comprises three zinc finger domains. In one embodiment, the artificial polypeptide comprises four zinc finger domains.

The zinc finger domain(s) of the artificial polypeptide can be naturally-occurring zinc finger domains, or variants thereof. The naturally-occurring zinc finger domains can be domains from any eukaryotic zinc finger protein: for example, a fungal (e.g., yeast), plant, or animal protein (e.g., a mammalian protein, such as a human or murine protein).

The artificial polypeptides can include other features described herein.

In another aspect, the invention features a cell selected by a method, the method including: providing a plurality of prokaryotic cells, wherein each cell of the plurality comprises a nucleic acid encoding an artificial polypeptide, wherein the artificial polypeptide comprises a zinc finger domain, and wherein the artificial polypeptide differs among the cells of the plurality; and, identifying from the plurality a cell that has a trait that is altered relative to a reference cell. The reference cell, e.g., is a cell that does not include a nucleic acid encoding an artificial polypeptide, e.g., the reference cell is a parental cell from which the plurality of cells was made, or a derivative thereof.

The trait can be any detectable phenotype, e.g., a phenotype that can be observed, selected, inferred, and/or quantitated. The artificial polypeptide can be a chimeric polypeptide. An artificial polypeptide can include one or more features described herein.

In another aspect, the invention features a polypeptide including at least one zinc finger domain, wherein the DNA contacting residues of the zinc finger domain at positions −1, +2, +3, and +6 correspond to a motif selected from: RSHR, HSSR, ISNR, RDHT, QTHR, VSTR, QNTQ, and CSNR, and wherein the polypeptide regulates an endogenous prokaryotic gene and/or alters the phenotype of a prokaryotic cell.

The polypeptide can further include a second and third zinc finger domain, wherein the DNA contacting residues of the first, second, and third domains at positions −1, +2, +3, and +6 of each domain respectively correspond to the motifs RSHR, HSSR, and ISNR.

The polypeptide can further include a second and third zinc finger domain, wherein the DNA contacting residues of the first, second, and third domains at positions −1, +2, +3, and +6 of each domain respectively correspond to the motifs ISNR, RDHT, and QTHR.

The polypeptide can further include a fourth zinc finger domain, wherein the DNA contacting residues of the fourth domain at positions −1, +2, +3, and +6 of correspond to the motif VSTR.

The polypeptide can further include a second and third zinc finger domain, wherein the DNA contacting residues of the first, second, and third domains at positions −1, +2, +3, and +6 of each domain respectively correspond to the motifs QNTQ, CSNR, and ISNR.

In another aspect, the invention feature a polypeptide including at least one zinc finger domain, wherein the DNA contacting residues of the zinc finger domain at positions −1, +2, +3, and +6 correspond to a motif selected from: QSHV, VSNV, QSNK, RDHT, QTHR, QSSR, WSNR, VSNV, RSHR, DSAR, QTHQ, RSHR, QSNR, and CSNR, and wherein the polypeptide regulates an endogenous prokaryotic gene and/or alters the phenotype of a prokaryotic cell.

In one embodiment, the polypeptide further includes a second, third, and fourth zinc finger domain, wherein the DNA contacting residues of the first, second, third, and fourth domains at positions −1, +2, +3, and +6 of each domain respectively correspond to the motifs QSHV, VSNV, QSNK, and QSNK.

In one embodiment, the polypeptide further includes a second, third, and fourth zinc finger domain, wherein the DNA contacting residues of the first, second, third, and fourth domains at positions −1, +2, +3, and +6 of each domain respectively correspond to the motifs RDHT, QSHV, QTHR1, and QSSR.

In one embodiment, the polypeptide further includes a second, third, and fourth zinc finger domain, wherein the DNA contacting residues of the first, second, third, and fourth domains at positions −1, +2, +3, and +6 of each domain respectively correspond to the motifs WSNR, QSHV, VSNV, and QSHV.

In one embodiment, the polypeptide further includes a second, third, and fourth zinc finger domain, wherein the DNA contacting residues of the first, second, third, and fourth domains at positions −1, +2, +3, and +6 of each domain respectively correspond to the motifs QTHR, RSHR, QTHR, and QTHR.

In one embodiment, the polypeptide further includes a second, third, and fourth zinc finger domain, wherein the DNA contacting residues of the first, second, third, and fourth domains at positions −1, +2, +3, and +6 of each domain respectively correspond to the motifs DSAR, RDHT, QSHV, and QTHR.

In one embodiment, the polypeptide further includes a second, third, and fourth zinc finger domain, wherein the DNA contacting residues of the first, second, third, and fourth domains at positions −1, +2, +3, and +6 of each domain respectively correspond to the motifs QTHQ, RSHR, QTHR, and QTHR.

In one embodiment, the polypeptide further includes a second, third, and fourth zinc finger domain, wherein the DNA contacting residues of the first, second, third, and fourth domains at positions −1, +2, +3, and +6 of each domain respectively correspond to the motifs QSHV, VSNV, QSNR, and CSNR.

In one embodiment, the polypeptide further includes a second, third, and fourth zinc finger domain, wherein the DNA contacting residues of the first, second, third, and fourth domains at positions −1, +2, +3, and +6 of each domain respectively correspond to the motifs VSNV, QTHR, QSSR, and RDHT.

In one embodiment, the polypeptide further includes a second, third, and fourth zinc finger domain, wherein the DNA contacting residues of the first, second, third, and fourth domains at positions −1, +2, +3, and +6 of each domain respectively correspond to the motifs RDHT, QSHV, QTHR, and QSNR.

In one embodiment, the polypeptide further includes a second, third, and fourth zinc finger domain, wherein the DNA contacting residues of the first, second, third, and fourth domains at positions −1, +2, +3, and +6 of each domain respectively correspond to the motifs DSAR, RDHT, QSNK, and QTHR.

In another aspect, the invention features an isolated nucleic acid encoding an artificial polypeptide described herein.

In another aspect, the invention features a bacterial nucleic acid expression vector encoding an artificial polypeptide described herein.

In another aspect, the invention features a method of producing a polypeptide, the method including: providing a prokaryotic cell, wherein the cell expresses an artificial polypeptide comprising a zinc finger domain, and wherein the artificial polypeptide binds to a target DNA site in a gene, culturing the cell under conditions that permit production of the polypeptide at a level higher or lower (e.g., at least two, three, five, ten, or a hundred fold) than the level produced by an identical cell that includes the gene but not the artificial polypeptide, and detecting the polypeptide produced by the cell and/or purifying the polypeptide from the cell and/or from the medium that surrounds the cell. The polypeptide can be an endogenous or heterologous polypeptide. Production of the polypeptide by the cell can be directly or indirectly regulated by the artificial polypeptide. The method can further include introducing the cell into a subject. The method can further include formulating the polypeptide with a pharmaceutically acceptable carrier.

In another aspect, the invention features a method of preparing a modified prokaryotic cell, the method including providing a nucleic acid library that includes a plurality of nucleic acids, each encoding a different artificial polypeptide, each polypeptide including at least two zinc finger domains; identifying a first and a second member of the library which alters a given trait of a cell; and preparing a cell that can express first and second polypeptides, the first and second polypeptides being encoded respectively by the first and second identified library members. The method can also be extended to additional member, e.g., a third member. The method can further include evaluating the given trait for the prepared cell. The method can include other features described herein.

In another aspect, the method includes a method of producing a cellular product. The method includes providing a modified cell that includes a nucleic acid encoding an artificial polypeptide; maintaining the modified cell under conditions in which the artificial polypeptide is produced; and recovering a product produced by the cultured cell, wherein the product is other than the artificial polypeptide. For example, the artificial polypeptide can confer stress resistance, or another property described herein, e.g., altered protein production, altered metabolite production, and so forth. For example, the artificial polypeptide includes at least two zinc finger domains. One or more of the zinc finger domains can be naturally occurring, e.g., a naturally occurring domain in Table 3. Exemplary artificial polypeptides include polypeptides that have one or more consecutive motifs (e.g., at least two, three or four consecutive motifs, or at least three motifs in the same pattern, including non-consecutive patterns) as described herein.

Exemplary products include a metabolite or a protein (e.g., an endogenous or heterologous protein. For example, the modified cell further includes a second nucleic acid encoding a heterologous protein, and the heterologous protein participates in production of the metabolite. The modified cell can be maintained at a temperature between 20° C. and 40° C. or greater than 37° C. In one embodiment, the modified cell is maintained under conditions which would inhibit the growth of a substantially identical cell that lacks the artificial polypeptide.

In another aspect, the invention features an artificial polypeptide that alters sensitivity of a cell expressing the artificial polypeptide to a toxic agent (e.g., a catabolite of the cell or a chemical) relative to an identical cell that does not express the artificial polypeptide. The sensitivity can be increased or decreased. Exemplary artificial polypeptides include polypeptides that have one or more zinc finger domains, e.g., zinc finger domains including motifs as described herein.

With respect to all methods described herein, a library of nucleic acids that encode chimeric zinc finger proteins can be used. The term “library” refers to a physical collection of similar, but non-identical biomolecules. The collection can be, for example, together in one vessel or physically separated (into groups or individually) in separate vessels or on separate locations on a solid support. Duplicates of individual members of the library may be present in the collection. A library can include at least 10, 10², 10³, 10⁵, 10⁷, or 10⁹ different members, or fewer than 10¹³, 10¹², 10¹⁰, 10⁹, 10⁷, 10⁵, or 10³ different members.

A first exemplary library includes a plurality of nucleic acids, each nucleic acid encoding a polypeptide comprising at least a first, second, and third zinc finger domains. As used herein, “first, second and third” denotes three separate domains that can occur in any order in the polypeptide: e.g., each domain can occur N-terminal or C-terminal to either or both of the others. The first zinc finger domain varies among nucleic acids of the plurality. The second zinc finger domain varies among nucleic acids of the plurality. At least 10 different first zinc finger domains are represented in the library. In one implementation, at least 0.5, 1, 2, 5%, 10%, or 25% of the members of the library binds at least one target site with a dissociation constant of no more than 7, 5, 3, 2, 1, 0.5, or 0.05 nM. The first and second zinc finger domains can be from different naturally-occurring proteins or are positioned in a configuration that differs from their relative positions in a naturally-occurring protein. For example, the first and second zinc finger domains may be adjacent in the polypeptide, but may be separated by one or more intervening zinc finger domains in a naturally occurring protein.

A second exemplary library includes a plurality of nucleic acids, each nucleic acid encoding a polypeptide that includes at least first and second zinc finger domains. The first and second zinc finger domains of each polypeptide (1) are identical to zinc finger domains of different naturally occurring proteins (and generally do not occur in the same naturally occurring protein or are positioned in a configuration that differs from their relative positions in a naturally-occurring protein), (2) differ by no more than four, three, two, or one amino acid residues from domains of naturally occurring proteins, or (3) are non-adjacent zinc finger domains from a naturally occurring protein. Identical zinc finger domains refer to zinc finger domains that are identical at each amino acid from the first metal coordinating residue (typically cysteine) to the last metal coordinating residue (typically histidine). The first zinc finger domain varies among nucleic acids of the plurality, and the second zinc finger domain varies among nucleic acids of the plurality. The naturally occurring protein can be any eukaryotic zinc finger protein: for example, a fungal (e.g., yeast), plant, or animal protein (e.g., a mammalian protein, such as a human or murine protein). Each polypeptide can further include a third, fourth, fifth, and/or sixth zinc finger domain. Each zinc finger domain can be a mammalian, e.g., human, zinc finger domain.

Other types of libraries can also be used, e.g., including mutated zinc finger domains.

In some embodiments, a library of nucleic acids encoding zinc finger proteins or a library of such proteins themselves can include members with different regulatory domains. For example, the library can include at least 10% of members with an activation domain, and at least another 10% of members with a repression domain. In another example, at least 10% have an activation domain or repression domain; another at least 10% has no regulatory domain. In still another example, some include an activation domain; others, a repression domain; still others, no regulatory domain at all. Other percentages, e.g., at least 20, 25, 30, 40, 50, 60% can also be used.

The term “gene” refers to coding and noncoding DNA sequence associated with the expression of a particular polypeptide. A gene includes, e.g., exonic sequences, intronic sequences, promoter, enhancer, and other regulatory sequences.

As used herein, the “dissociation constant” refers to the equilibrium dissociation constant of a polypeptide for binding to a 28-basepair double-stranded DNA that includes one 9-basepair target site. The dissociation constant is determined by gel shift analysis using a purified protein that is bound in 20 mM Tris pH 7.7, 120 mM NaCl, 5 mM MgCl₂, 20 μM ZnSO₄, 10% glycerol, 0.1% Nonidet P-40, 5 mM DTT, and 0.10 mg/mL BSA (bovine serum albumin) at room temperature. Additional details are provided in Example 10 and Rebar and Pabo (1994) Science 263:671-673.

As used herein, the term “screen” refers to a process for evaluating members of a library to find one or more particular members that have a given property. In a direct screen, each member of the library is evaluated. For example, each cell is evaluated to determine if it is extending neurites. In another type of screen, termed a “selection,” each member is not directly evaluated. Rather the evaluation is made by subjecting the members of the library to conditions in which only members having a particular property are retained. Selections may be mediated by survival (e.g., drug resistance) or binding to a surface (e.g., adhesion to a substrate). Such selective processes are encompassed by the term “screening.”

The term “base contacting positions,” “DNA contacting positions,” or “nucleic acid contacting positions” refers to the four amino acid positions of a zinc finger domain that structurally correspond to the positions of amino acids arginine 73, aspartic acid 75, glutamic acid 76, and arginine 79 of ZIF268.

| Glu Arg Pro Tyr Ala Cys Pro Val Glu Ser Cys Asp Arg Arg Phe Ser | (SEQ ID NO:65) |
|---|---|
| 1 5 10 15 | |
| Arg Ser Asp Glu Leu Thr Arg His Ile Arg Ile His Thr Gly Gln Lys | |
| 20 25 30 | |
| Pro Phe Gln Cys Arg Ile Cys Met Arg Asn Phe Ser Arg Ser Asp His | |
| 35 40 45 | |
| Leu Thr Thr His Ile Arg Thr His Thr Gly Glu Lys Pro Phe Ala Cys | |
| 50 55 60 | |
| Asp Ile Cys Gly Arg Lys Phe Ala Arg Ser Asp Glu Arg Lys Arg His | |
| 65 70 75 80 | |
| Thr Lys Ile His Leu Arg Gln Lys Asp | |
| 85 | |

These positions are also referred to as positions −1, 2, 3, and 6, respectively. To identify positions in a query sequence that correspond to the base contacting positions, the query sequence is aligned to the zinc finger domain of interest such that the cysteine and histidine residues of the query sequence are aligned with those of finger 3 of Zif268. The ClustalW WWW Service at the European Bioinformatics Institute (Thompson et al. (1994) Nucleic Acids Res. 22:4673-4680) provides one convenient method of aligning sequences.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; a group of amino acids having acidic side chains is aspartic acid and glutamic acid; and a group of amino acids having sulfur-containing side chains is cysteine and methionine. Depending on circumstances, amino acids within the same group may be interchangeable. Some additional conservative amino acids substitution groups are: valine-leucine-isoleucine; phenylalanine-tyrosine; lysine-arginine; alanine-valine; aspartic acid-glutamic acid; and asparagine-glutamine.

The term “heterologous polypeptide” or “artificial polypeptide” refers either to a polypeptide with a non-naturally occurring sequence (e.g., a hybrid polypeptide) or a polypeptide with a sequence identical to a naturally occurring polypeptide but present in a milieu in which it does not naturally occur. For example, the fusion of two naturally occurring polypeptides that are not fused together in Nature results in an artificial polypeptide in which one polypeptide is heterologous to the other.

The terms “hybrid” and “chimera” refer to a non-naturally occurring polypeptide that comprises amino acid sequences derived from either (i) at least two different naturally occurring sequences, or non-contiguous regions of the same naturally occurring sequence, wherein the non-contiguous regions are made contiguous in the hybrid; (ii) at least one artificial sequence (i.e., a sequence that does not occur naturally) and at least one naturally occurring sequence; or (iii) at least two artificial sequences (same or different). Examples of artificial sequences include mutants of a naturally occurring sequence and de novo designed sequences. An “artificial sequence” is not present among naturally occurring sequences. With respect to any artificial sequence (e.g., protein or nucleic acid) described herein, the invention also refers to a sequence with the same elements, but which is not present in each of the following organisms whose genomes are sequenced: Homo sapiens, Mus musculus, Arabidopsis thaliana, Drosophila melanogaster, Escherichia coli, Saccharomyces cerevisiae, and Oryza sativa. A molecule with such a sequence can be expressed as a heterologous molecule in a cell of one of the afore-mentioned organisms.

The invention also includes sequences (not necessarily termed “artificial”) which are made by a method described herein, e.g., a method of joining nucleic acid sequences encoding different zinc finger domains or a method of phenotypic screening. The invention also features a cell that includes such a sequence.

As used herein, the term “hybridizes under stringent conditions” refers to conditions for hybridization in 6× sodium chloride/sodium citrate (SSC) at 45° C., followed by two washes in 0.2× SSC, 0.1% SDS at 65° C.

The term “binding preference” refers to the discriminative property of a polypeptide for selecting one nucleic acid binding site relative to another. For example, when the polypeptide is limiting in quantity relative to two different nucleic acid binding sites, a greater amount of the polypeptide will bind the preferred site relative to the other site in an in vivo or in vitro assay described herein.

A “reference cell” refers to any cell of interest. In one example, the reference cell is a parental cell for a cell that expresses a zinc finger protein, e.g., a cell that is substantially identical to the zinc finger protein expressing cell, but which does not produce the zinc finger protein.

A “transformed” or “transfected” cell refers to a cell that includes a heterologous nucleic acid. The cell can be made by introducing (e.g., transforming, transfecting, or infecting, e.g., using a viral particle) a nucleic acid into the cell or the cell can be a progeny or derivative of a cell thus made.

Among other advantages, many of the methods and compositions relate to the identification and use of new and useful zinc finger proteins for regulating gene expression in prokaryotic cells. Endogenous genes can be either up- or down-regulated using modular zinc finger proteins. Even without a transcriptional regulatory domain (e.g., a repression or activation domain), zinc finger proteins can be potent modulators of gene expression. It is possible to screen a plurality of cells expressing zinc finger proteins with different DNA binding specificities, in order to identify cells having altered traits due to altered gene expression. Moreover, gene expression in prokaryotes can be finely regulated, by regulating expression of the zinc finger proteins. Depending on the DNA-binding affinity, chimeric polypeptides can cause a range of effects, e.g., moderate to strong activation and repression. This may lead to diverse phenotypes that are not necessarily obtained by completely inactivation or high level over-expressed of a particular target gene.

Methods described herein do not require a priori information (e.g., genome sequence) of the cell in order to identify useful chimeric proteins. Artificial chimeric proteins can be used as a tool to dissect pathways within a cell. For example, target genes responsible for the phenotypic changes in selected clones can be identified, e.g., as described herein. A zinc finger protein may mimic the function of a master regulatory protein, such as a master regulatory transcription factor. For example, the zinc finger protein may bind to the same site as the master regulatory, or to an overlapping site. The level of gene expression change, thus the extent of the phenotype generated by ZFP-TF, can be precisely controlled by altering the expression level of zinc finger protein in cells.

All patents, patent applications, and references cited herein are incorporated by reference in their entirety. The following patent applications: WO 01/60970 (Kim et al.); U.S. Ser. No. 60/338,441, filed Dec. 7, 2001; U.S. Ser. No. 60/313,402, filed Aug. 17, 2001; U.S. Ser. No. 60/374,355, filed Apr. 22, 2002; U.S. Ser. No. 60/376,053, filed Apr. 26, 2002; U.S. Ser. No. 60/400,904, filed Aug. 2, 2002; U.S. Ser. No. 60/401,089, filed Aug. 5, 2002; and U.S. Ser. No. 10/223,765, filed Aug. 19, 2002, are expressly incorporated by reference in their entirety for all purposes. The details of one or more embodiments of the invention are set forth in the accompanying drawings and the description below. Any feature described herein can be used in combination with another compatible feature also described herein. Other features, objects, and advantages of the invention will be apparent from the description and drawings, and from the claims.

### DESCRIPTION OF THE DRAWINGS

FIGS. 1A, 1B, and 1C are a set of pictures depicting phenotypic changes in E. coli induced by expression of artificial zinc finger proteins. FIG. 1A depicts growth of cells on LB plates in the presence or absence of 1.5% hexane. Clones H1, H2, and H3 expressed zinc finger proteins. Control cells (C; E. coli cells transformed with pZL1) did not express zinc finger proteins. FIG. 1B depicts growth of heat-shocked, and untreated cells on LB plates. Selected clones (T1 to T10) expressed zinc finger proteins. Control cells (C; E. coli cells transformed with pZL1) did not express zinc finger proteins. FIG. 1C depicts growth of control cells (C; E. coli cells transformed with pZL1), cells expressing the T9 zinc finger protein (T9), and cells expressing a mutated version of T9 (T9-M) on LB plates. An arginine residue in the QTHR1 zinc finger domain of the T9 protein was mutated to alanine to produce T9-M. Cells were heat-shocked or untreated. In FIG. 1A. and FIG. 1B, the triangles drawn above of each panel indicate 10-fold serial dilutions (1:1 to 1:10,000, left to right) of spotted cells.

FIGS. 2A, 2B, and 2C. Identification of a Target Gene Regulated by Zinc Finger proteins

FIG. 2A (left panel) depicts growth of control cells (C; E. coli cells transformed with pZL1), cells transformed with zinc finger protein T9, and cells containing a disruption in the UbiX gene (ubiX) on LB plates. Cells were heat-shocked or untreated. The triangles drawn above of each panel indicate 10-fold serial dilutions (1:1 to 1:10,000, left to right) of spotted cells. FIG. 2A (right panel) is a graph depicting the percent survival of heat-shocked control cells (C; E. coli cells transformed with pZL1), T9-transformed cells, and cells containing a disruption in the ubiX gene (ubiX). FIG. 2B is a graph depicting the relative level of UbiX transcripts in control and T9-expressing cells. FIG. 2C is a schematic diagram depicting the interaction T9-ZFP with potential binding sites located in the UbiX promoter. The position of potential binding sites relative to the transcription start site is indicated. Binding of T9-ZFP to the position was confirmed by immuno-precipitation.

Like reference symbols in the various drawings indicate like elements.

### DETAILED DESCRIPTION

The invention is based, in part, on the discovery that zinc finger proteins can regulate gene expression in prokaryotic organisms. Zinc finger proteins (e.g., zinc finger proteins that include eukaryotic zinc finger domains) can modulate expression of endogenous genes in prokaryotes.

Expression of libraries of zinc finger proteins in prokaryotic cells can allow the identification of zinc finger proteins that alter a phenotype of the cells. Furthermore, expression of these proteins enables the identification of gene products (e.g., endogenously-expressed gene products), the modulation of which alters a phenotype of the cells.

In one embodiment, a nucleic acid library that encodes artificial polypeptides which include random chimeras of zinc finger domains is transformed into prokaryotic cells (e.g., E. coli cells). Nucleic acids of the library are expressed in the cells. The cells are evaluated for a phenotype of interest, and cells in which the phenotype is altered relative to a control are isolated. The library nucleic acids in such cells are recovered, and the zinc finger protein encoded by such recovered nucleic acids can be further characterized, utilized, or modified. The target DNA site bound by the zinc finger protein can also be recovered and characterized. In one embodiment, the genes that include the target DNA sites are identified, thereby revealing genes involved in modulation of the phenotype of interest.

Chimeric zinc finger proteins that include, one, two, three, four, or more zinc finger domains can be used to regulate gene expression in prokaryotic cells. These zinc finger proteins can include two or more naturally-occurring zinc finger proteins.

Zinc finger proteins may also be engineered to recognize a target DNA site in a prokaryotic cell. Useful target sites include sites in a regulatory region of the target gene or within 1 kb or 500 bp of a regulatory region of a target gene. For example, the target site can be within 1 kb or 500 bp of a transcriptional start site of a gene. One method for designing a zinc finger protein includes parsing target sites into 3 or 4 basepair sequences that can be recognized by an individual zinc finger domain. Then a nucleic acid is constructed which includes a sequence that encodes a protein that has consecutive zinc finger domains corresponding to the parsed elements. A plurality of different nucleic acids that encode candidate proteins is constructed and expressed in a host cell. The expression of the target gene is evaluated to identify one or more of the candidates that is able to regulate expression of the target gene.

In one aspect of the invention, a library of nucleic acids that encode different artificial, chimeric polypeptides is screened to identify a chimeric protein that alters a phenotypic trait of a prokaryotic cell. The artificial polypeptide can be identified without a priori knowledge of a particular target gene or pathway.

Library Construction

The nucleic acid library is constructed so that it includes nucleic acids that each encode and can express an artificial polypeptide that is a chimera of one or more structural domains (e.g., zinc finger domains). The zinc finger domains are nucleic acid binding domains that can vary in specificity such that the library encodes a population of proteins with different binding specificities.

Zinc fingers. Zinc fingers are small polypeptide domains of approximately 30 amino acid residues in which there are four amino acids, either cysteine or histidine, appropriately spaced such that they can coordinate a zinc ion (For reviews, see, e.g., Klug and Rhodes, (1987) Trends Biochem. Sci. 12:464-469(1987); Evans and Hollenberg, (1988) Cell 52:1-3; Payre and Vincent, (1988) FEBS Lett. 234:245-250; Miller et al., (1985) EMBO J. 4:1609-1614; Berg, (1988) Proc. Natl. Acad. Sci. U.S.A. 85:99-102; Rosenfeld and Margalit, (1993) J. Biomol. Struct. Dyn. 11:557-570). Hence, zinc finger domains can be categorized according to the identity of the residues that coordinate the zinc ion, e.g., as the Cys₂-His₂ class, the Cys₂-Cys₂ class, the Cys₂-CysHis class, and so forth. The zinc coordinating residues of Cys₂-His₂ zinc fingers are typically spaced as follows: Xₐ-X-C-X₂₋₅-C-X₃-Xₐ-X₅-ψ-X₂-H-X₃₋₅-H (SEQ ID NO:66), where ψ (psi) is a hydrophobic residue (Wolfe et al., (1999) Annu. Rev. Biophys. Biomol. Struct. 3:183-212), wherein “X” represents any amino acid, wherein Xₐ is phenylalanine or tyrosine, the subscript indicates the number of amino acids, and a subscript with two hyphenated numbers indicates a typical range of intervening amino acids. Typically, the intervening amino acids fold to form an anti-parallel β-sheet that packs against an α-helix, although the anti-parallel β-sheets can be short, non-ideal, or non-existent. The fold positions the zinc-coordinating side chains so they are in a tetrahedral conformation appropriate for coordinating the zinc ion. The base contacting residues are at the N-terminus of the finger and in the preceding loop region.

For convenience, the primary DNA contacting residues of a zinc finger domain are numbered: −1, 2, 3, and 6 based on the following example:

−1 1 2 3 4 5 6

Xₐ-X-C-X₂₋₅-C-X₃-Xₐ-X-C-X-S-N-X_{b}-X-R-H-X₃₋₅-H (SEQ ID NO: 123),

where Xₐ is typically phenylalanine or tyrosine, and X_{b} is typically a hydrophobic residue. As noted in the example above, the DNA contacting residues are Cys (C), Ser (S), Asn (N), and Arg (R). The above motif can be abbreviated CSNR As used herein, such abbreviation refers to a class of sequences which include a domain corresponding to the motif as wells as a species whose sequence includes a particular polypeptide sequence, typically a sequence listed in Table 1 or Table 3 that conforms to the motif. Where two sequences in Table 1 Table 3 have the same motif, a number may be used to indicate the sequence.

A zinc finger protein typically consists of a tandem array of three or more zinc finger domains. For example, zinc finger domains whose motifs are listed consecutively are not interspersed with other folded domains, but may include a linker, e.g., a flexible linker described herein between domains. For an implementation that includes a specific zinc finger protein or array thereof described herein, the invention also features a related implementation that includes a corresponding zinc finger protein or array thereof having an array with zinc fingers that have the same DNA contacting residues as the specific zinc finger protein or array thereof. The corresponding zinc finger protein may differ by at least one, two, three, four, or five amino acids from the disclosed specific zinc finger protein, e.g., at an amino acid position that is not a DNA contacting residue. Other related implementations include a corresponding protein that has at least one, two, or three zinc fingers that have the same DNA contacting residues, e.g., in the same order.

The zinc finger domain (or “ZFD”) is one of the most common eukaryotic DNA-binding motifs, found in species from yeast to higher plants and to humans. By one estimate, there are at least several thousand zinc finger domains in the human genome alone, possibly at least 4,500. Zinc finger domains can be isolated from zinc finger proteins. Non-limiting examples of zinc finger proteins include CF2-II, Kruppel, WT1, basonuclin, BCL-6/LAZ-3, erythroid Kruppel-like transcription factor, Sp1, Sp2, Sp3, Sp4, transcriptional repressor YY1, EGR1/Krox24, EGR2/Krox20, EGR3/Pilot, EGR4/AT133, Evi-1, GLI1, GLI2, GLI3, HIV-EP1/ZNF40, HIV-EP2, KR1, ZfX, ZfY, and ZNF7.

Computational methods described below can be used to identify all zinc finger domains encoded in a sequenced genome or in a nucleic acid database. Any such zinc finger domain can be utilized. In addition, artificial zinc finger domains have been designed, e.g., using computational methods (e.g., Dahiyat and Mayo, (1997) Science 278:82-7).

It is also noteworthy that at least some zinc finger domains bind to ligands other than DNA, e.g., RNA or protein. Thus, a chimera of zinc finger domains or of a zinc finger domain and another type of domain can be used to recognize a variety of target compounds, not just DNA.

WO 01/60970, U.S. Ser. No. 60/374,355, filed Apr. 22, 2002, and U.S. Ser. No. 10/223,765, filed Aug. 19, 2002, describe exemplary zinc finger domains which can be used to construct an artificial zinc finger protein. See also the Table 3, below.

A variety of other structural domains are known to bind nucleic acids with high affinity and high specificity. For reviews of structural motifs which recognize double stranded DNA, see, e.g., Pabo and Sauer (1992) Annu. Rev. Biochem. 61:1053-95; Patikoglou and Burley (1997) Annu. Rev. Biophys. Biomol. Struct. 26:289-325; Nelson (1995) Curr Opin Genet Dev. 5:180-9.

Identification of zinc finger domains. A variety of methods can be used to identify zinc finger domains. Nucleic acids encoding identified domains are used to construct the nucleic acid library. Further, nucleic acid encoding these domains can also be varied (e.g., mutated) to provide additional domains that are encoded by the library.

Computational Methods. To identify additional naturally-occurring structural domains (e.g., zinc finger domains), the amino acid sequence of a known zinc finger domain can be compared to a database of known sequences, e.g., an annotated database of protein or nucleic acid sequences. In another implementation, databases of uncharacterized sequences, e.g., unannotated genomic, EST or full-length cDNA sequence; of characterized sequences, e.g., SwissProt or PDB; and of domains, e.g., Pfam, ProDom (Corpet et al. (2000) Nucleic Acids Res. 28:267-269), and SMART (Simple Modular Architecture Research Tool, Letunic et al. (2002) Nucleic Acids Res 30, 242-244) can provide a source of zinc finger domain sequences. Nucleic acid sequence databases can be translated in all six reading frames for the purpose of comparison to a query amino acid sequence. Nucleic acid sequences that are flagged as encoding candidate nucleic acid binding domains can be amplified from an appropriate nucleic acid source, e.g., genomic DNA or cellular RNA. Such nucleic acid sequences can be cloned into an expression vector. The procedures for computer-based domain identification can be interfaced with an oligonucleotide synthesizer and robotic systems to produce nucleic acids encoding the domains in a high-throughput platform. Cloned nucleic acids encoding the candidate domains can also be stored in a host expression vector and shuttled easily into an expression vector, e.g., into a translational fusion vector with other domains (of a similar or different type), either by restriction enzyme mediated subcloning or by site-specific, recombinase mediated subcloning (see U.S. Pat. No. 5,888,732). The high-throughput platform can be used to generate multiple microtitre plates containing nucleic acids encoding different candidate chimeras.

Detailed methods for the identification of domains from a starting sequence or a profile are well known in the art. See, for example, Prosite (Hofmann et al., (1999) Nucleic Acids Res. 27:215-219), FASTA, BLAST (Altschul et al., (1990) J. Mol. Biol. 215:403-10.), etc. A simple string search can be done to find amino acid sequences with identity to a query sequence or a query profile, e.g., using Perl to scan text files. Sequences so identified can be about 30%, 40%, 50%, 60%, 70%, 80%, 90%, or greater identical to an initial input sequence.

Domains similar to a query domain can be identified from a public database, e.g., using the XBLAST programs (version 2.0) of Altschul et al., (1990) J. Mol. Biol. 215:403-10. For example, BLAST protein searches can be performed with the XBLAST parameters as follows: score=50, wordlength=3. Gaps can be introduced into the query or searched sequence as described in Altschul et al., (1997) Nucleic Acids Res. 25(17): 3389-3402. Default parameters for XBLAST and Gapped BLAST programs are available at National Center for Biotechnology Information (NCBI), National Institutes of Health, Bethesda Md.

The Prosite profiles PS00028 and PS50157 can be used to identify zinc finger domains. In a SWISSPROT release of 80,000 protein sequences, these profiles detected 3189 and 2316 zinc finger domains, respectively. Profiles can be constructed from a multiple sequence alignment of related proteins by a variety of different techniques. Gribskov and co-workers (Gribskov et al., (1990) Meth. Enzymol. 183:146-159) utilized a symbol comparison table to convert a multiple sequence alignment supplied with residue frequency distributions into weights for each position. See, for example, the PROSITE database and the work of Luethy et al., (1994) Protein Sci. 3:139-1465.

Hidden Markov Models (HMM's) representing a DNA binding domain of interest can be generated or obtained from a database of such models, e.g., the Pfam database, release 2.1. A database can be searched, e.g., using the default parameters, with the HMM in order to find additional domains (see, e.g., Bateman et al. (2002) Nucleic Acids Research 30:276-280). Alternatively, the user can optimize the parameters. A threshold score can be selected to filter the database of sequences such that sequences that score above the threshold are displayed as candidate domains. A description of the Pfam database can be found in Sonhammer et al., (1997) Proteins 28(3): 405-420, and a detailed description of HMMs can be found, for example, in Gribskov et al., (1990) Meth. Enzymol. 183:146-159; Gribskov et al., (1987) Proc. Natl. Acad. Sci. USA 84:4355-4358; Krogh et al., (1994) J. Mol. Biol. 235:1501-1531; and Stultz et al., (1993) Protein Sci. 2:305-314.

The SMART database of HMM's (Simple Modular Architecture Research Tool, Schultz et al., (1998) Proc. Natl. Acad. Sci. USA 95:5857 and Schultz et al., (2000) Nucl. Acids Res 28:231) provides a catalog of zinc finger domains (ZnF_C₂H₂; ZnF_C2C2; ZnF_C2HC; ZnF_C3H1; ZnF_C4; ZnF_CHCC; ZnF_GATA; and ZNF_NFX) identified by profiling with the hidden Markov models of the HMMer2 search program (Durbin et al., (1998) Biological sequence analysis: probabilistic models of proteins and nucleic acids. Cambridge University Press).

Hybridization-based Methods. A collection of nucleic acids encoding various forms of a zinc finger domain can be analyzed to profile sequences encoding conserved amino- and carboxy-terminal boundary sequences. Degenerate oligonucleotides can be designed to hybridize to sequences encoding such conserved boundary sequences. Moreover, the efficacy of such degenerate oligonucleotides can be estimated by comparing their composition to the frequency of possible annealing sites in known genomic sequences. If desired, multiple rounds of design can be used to optimize the degenerate oligonucleotides.

Comparison of known Cys₂-His₂ zinc fingers, for example, revealed a common sequence in the linker region between adjacent fingers in natural sequence (Agata et al., (1998) Gene 213:55-64). Degenerate oligonucleotides that anneal to nucleic acid encoding the conserved linker region were used to amplify a plurality of zinc finger domains. The amplified nucleic acid encoding the domains can be used to construct nucleic acids that encode a chimeric array of zinc fingers.

Nucleic Acids Encoding Zinc Finger Domains

Nucleic acids that are used to assemble the library can be obtained by a variety of methods. Some component nucleic acids of the library can encode naturally occurring zinc finger domains. In addition, some component nucleic acids are variants that are obtained by mutation or other randomization methods. The component nucleic acids, typically encoding just a single domain, can be joined to each other to produce nucleic acids encoding a fusion of the different zinc finger domains.

Isolation of a natural repertoire of domains. A library of domains can be constructed by isolation of nucleic acid sequences encoding domains from genomic DNA or cDNA of eukaryotic organisms such as yeasts or humans. Multiple methods are available for doing this. For example, a computer search of available amino acid sequences can be used to identify the domains, as described above. A nucleic acid encoding each domain can be isolated and inserted into a vector appropriate for the expression in cells, e.g., a vector containing a promoter, an activation domain, and a selectable marker. In another example, degenerate oligonucleotides that hybridize to a conserved motif are used to amplify, e.g., by PCR, a large number of related domains containing the motif. For example, Kruppel-like Cys₂His₂ zinc fingers can be amplified by the method of Agata et al., (1998) Gene 213:55-64. This method also maintains the naturally occurring zinc finger domain linker peptide sequences, e.g., sequences with the pattern: Thr-Gly-(Glu/Gln)-(Lys/Arg)-Pro-(Tyr/Phe) (SEQ ID NO: 122). Moreover, screening a collection limited to domains of interest, unlike screening a library of unselected genomic or cDNA sequences, significantly decreases library complexity and reduces the likelihood of missing a desirable sequence due to the inherent difficulty of completely screening large libraries.

The human genome contains numerous zinc finger domains, many of which are uncharacterized and unidentified. It is estimated that there are thousands of genes encoding proteins with zinc finger domains (Pellegrino and Berg, (1991) Proc. Natl. Acad. Sci. USA 88:671-675). These human zinc finger domains represent an extensive collection of diverse domains from which novel DNA-binding proteins can be constructed. Many exemplary human zinc finger domains are described in WO 01/60970, U.S. Ser. No. 60/374,355, filed Apr. 22, 2002, and U.S. Ser. No. 10/223,765, filed Aug. 19, 2002. See also Table 3 below.

TABLE 3
Exemplary Zinc Finger Domains

| | | | |
|---|---|---|---|
| | | SEQ ID | Target |
| ZFD | Amino acid sequence | NO: | subsite(s) |

| | | | |
|---|---|---|---|
| CSNR1 | YKCKQCGKAFGCPSNLRRHGRTH | 67 | GAA > GAC > GAG |
| CSNR2 | YQCNICGKCFSCNSNLHRHQRTH | 68 | GAA > GAC > GAG |
| DSAR2 | YSCGICGKSFSDSSAKRRHCILH | 69 | GTC |
| DSCR | YTCSDCGKAFRDKSCLNRHRRTH | 70 | GCC |
| HSNK | YKCKECGKAFNHSSNFNKHHRIH | 71 | GAC |
| HSSR | FKCPVCGKAFRHSSSLVRHQRTH | 72 | GTT |
| ISNR | YRCKYCDRSFSISSNLQRHVRNIH | 73 | GAA > GAT > GAC |
| ISNV | YECDHCGKAFSIGSNLNVHRRIH | 74 | AAT |
| KSNR | YGCHLCGKAFSKSSNLRRHEMIH | 75 | GAG |
| QAHR | YKCKECGQAFRQRAHLIRHHKLH | 76 | GGA |
| QFNR | YKCHQCGKAFIQSFNLRRHERTH | 77 | GAG |
| QGNR | FQCNQCGASFTQKGNLLRHIKLH | 78 | GAA |
| QSHR1 | YACHLCGKAFTQSSHLRRHEKTH | 79 | GGA > GAA > AGA |
| QSHR2 | YKCGQCGKFYSQVSHLTRHQKIH | 80 | GGA |
| QSHR3 | YACHLCGKAFTQCSHLRRHEKTH | 81 | GGA > GAA |
| QSHR4 | YACHLCAKAFIQCSHLRRHEKTH | 82 | GGA > GAA |
| QSHR5 | YVCRECGRGFRQHSHLVRHKRTH | 83 | GGA > AGA > GAA > CGA |
| QSHT | YKCEECGKAFRQSSHLTTHKIIH | 84 | AGA, CGA > TGA > GGA |
| QSHV | YECDHCGKSFSQSSHLNVHKRTH | 85 | CGA > AGA > TGA |
| QSNI | YMCSECGRGFSQKSNLIIHQRTH | 86 | AAA, CAA |
| QSNK | YKCEECGKAFTQSSNLTKHKKIH | 87 | GAA > TAA > AAA |
| QSNR1 | FECKDCGKAFIQKSNLIRHQRTH | 88 | GAA |
| QSNR2 | YVCRECRRGFSQKSNLIRHQRTH | 89 | GAA |
| QSNR3 | YECEKCGKAFNQSSNLTRHKKSH | 90 | GAA |
| QSNV1 | YECNTCRKTFSQKSNLIVHQRTH | 91 | AAA > CAA |
| QSNV2 | YVCSKCGKAFTQSSNLTVHQKIH | 92 | AAA > CAA |
| QSNV3 | YKCDECGKNFTQSSNLIVHKRIH | 93 | AAA |
| QSNV4 | YECDVCGKTFTQKSNLGVHQRTH | 94 | AAA |
| QSNT | YECVQCGKGFTQSSNLITHQRVH | 95 | AAA |
| QSSR1 | YKCPDCGKSFSQSSSLIRHQRTH | 96 | GTA > GCA |
| QSSR2 | YECQDCGRAFNQNSSLGRHKRTH | 97 | GTA |
| QSSR3 | YECNECGKFFSQSSSLIRHRRSH | 98 | GTA > GCA |
| QSTR | YKCEECGKAFNQSSTLTRHKIVH | 99 | GTA > GCA |
| QSTV | YECNECGKAFAQNSTLRVHQRIH | 100 | ACA |
| QTHQ | YECHDCGKSFRQSTHLTQHRRIH | 101 | AGA > CGA, TGA |
| QTHR1 | YECHDCGKSFRQSTHLTRHRRIH | 102 | GGA > AGA, GAA |
| QTHR2 | HKCLECGKCFSQNTHLTRHQRT | 103 | GGA |
| RDER1 | YVCDVEGCTWKFARSDELNRHKKRH | 104 | GCG > GTG, GAC |
| RDER2 | YHCDWDGCGWKFARSDELTRHYRKH | 105 | GCG > GTG |
| RDER3 | YRCSWEGCEWRFARSDELTRHFRKH | 106 | GCG > GTG |
| RDER4 | FSCSWKGCERRFARSDELSRHRRTH | 107 | GCG > GTG |
| RDER5 | FACSWQDCNKKFARSDELARHYRTH | 108 | GCG |
| RDER6 | YHCNWDGCGWKFARSDELTRHYRKH | 109 | GCG > GTG |
| RDHR1 | FLCQYCAQRFGRKDHLTRHMKKSH | 110 | GAG, GGG |
| RDHT | FQCKTCQRKFSRSDHLKTHTRTH | 111 | AGG, CGG, GGG, TGG |
| RDKI | FACEVCGVRFTRNDKLKIHMRKH | 112 | GGG |
| RDKR | YVCDVEGCTWKFARSDKLNRHKKRH | 113 | GGG > AGG |
| RSHR | YKCMECGKAFNRRSHLTRHQRIH | 114 | GGG |
| RSNR | YICRKCGRGFSRKSNLIRHQRTH | 115 | GAG > GTG |
| RTNR | YLCSECDKCFSRSTNLIRHRRTH | 116 | GAG |
| SSNR | YECKECGKAFSSGSNFTRHQRIH | 117 | GAG > GAC |
| VSNV | YECDHCGKAFSVSSNLNVHRRIH | 118 | AAT > CAT > TAT |
| VSSR | YTCKQCGKAFSVSSSLRRHETTH | 119 | GTT > GTG > GTA |
| VSTR | YECNYCGKTFSVSSTLIRHQRIH | 120 | GCT > GCG |
| WSNR | YRCEECGKAFRWPSNLTRHKRIH | 121 | GGT > GGA |

If each zinc finger domain recognizes a unique 3- to 4-bp sequence, the total number of domains required to bind every possible 3- to 4-bp sequence is only 64 to 256 (4³ to 4⁴). It is possible that the natural repertoire of the human genome contains a sufficient number of unique zinc finger domains to span all possible recognition sites. These zinc finger domains are a valuable resource for constructing artificial chimeric DNA-binding proteins. A nucleic acid library can include nucleic acids encoding proteins that include naturally occurring zinc finger domains, artificial mutants of such domains, and combinations thereof.

Mutated Domains. In one implementation, the library includes nucleic acids encoding at least one structural domain that is an artificial variant of a naturally-occurring sequence. In one embodiment, such variant domains are assembled from a degenerate patterned library. In the case of a nucleic acid binding domains, positions in close proximity to the nucleic acid binding interface or adjacent to a position so located can be targeted for mutagenesis. A mutated test zinc finger domain, for example, can be constrained at any mutated position to a subset of possible amino acids by using a patterned degenerate library. Degenerate codon sets can be used to encode the profile at each position. For example, codon sets are available that encode only hydrophobic residues, aliphatic residues, or hydrophilic residues. The library can be selected for full-length clones that encode folded polypeptides. Cho et al. ((2000) J. Mol. Biol. 297(2): 309-19) provides a method for producing such degenerate libraries using degenerate oligonucleotides, and also provides a method of selecting library nucleic acids that encode full-length polypeptides. Such nucleic acids can be easily inserted into an expression plasmid, e.g., using convenient restriction enzyme cleavage sites.

Selection of the appropriate codons and the relative proportions of each nucleotide at a given position can be determined by simple examination of a table representing the genetic code, or by computational algorithms. For example, Cho et al., supra, describe a computer program that accepts a desired profile of protein sequence and outputs a preferred oligonucleotide design that encodes the sequence.

See also Zhang et al., (2000) J. Biol. Chem. 275:33850-33860; Rebar and Pabo (1994) Science 263:671-673; Segal (1999) Proc. Natl. Acad. Sci. USA 96:2758; Gogus et al., (1996) Proc. Natl. Acad. Sci. USA. 93:2159-2164; Drier et al., (2001) J. Biol. Chem. 276: 29466-29478; Liu et al. (2001) J. Biol. Chem. 276(14): 11323-11334; and Hsuetal., (1992) Science 257:1946-50 for some available zinc finger domains.

In one embodiment, a chimeric protein can include one or more of the zinc finger domains that have at least 18, 19, 20, 21, 22, 23, 24, or 25 amino acids that are identical to a zinc finger domain sequence in Table 1 or Table 3, or are at least 70, 75, 80, 85, 90, or 95% identical to a zinc finger domain sequence in Table 1 or Table 3. For example, the DNA contacting residues can be identical.

Construction of Chimeric Zinc Finger Proteins

A library of nucleic acids encoding diverse chimeric zinc finger proteins can be formed by serial ligation, e.g., as described in Example 1. The library can be constructed such that each nucleic acid encodes a protein that has at least three, four, or five zinc finger domains. In some implementations, particularly for large libraries, each zinc finger coding segment can be designed to randomly encode any one of a set of zinc finger domains. The set of zinc finger domains can be selected to represent domains with a range of specificities, e.g., covering 30, 40, 50 or more of the 64 possible 3-basepair subsites. The set can include at least about 12, 15, 20, 25, 30, 40 or 50 different zinc finger domains. Some or all of these domains can be domains isolated from naturally occurring proteins. Moreover, because there may be little or no need for more than one zinc finger domain for a given 3-basepair subsite, it may be possible to generate a library using a small number of component domains, e.g., less than 500, 200, 100, or even less than 64 total component domains.

One exemplary library includes nucleic acids that encode a chimeric zinc finger protein having three fingers and 30 possible domains at each finger position. In its fully represented form, this library includes 27,000 sequences (i.e., the result of 30³). The library can be constructed by serial ligation in which a nucleic acid from a pool of nucleic acids encoding all 30 possible domains is added at each step.

In one embodiment, the library can be stored as a random collection. In another embodiment, individual members can be isolated, stored at an addressable location (e.g., arrayed), and sequenced. After high throughput sequencing of 40 to 50 thousand constructed library members, missing chimeric combinations can be individually assembled in order to obtain complete coverage. Once arrayed, e.g., in microtitre plates, each individual member can be recovered later for further analysis, e.g., for a phenotypic screen. For example, equal amounts of each arrayed member can be pooled and then transformed into a cell. Cells with a desired phenotype are selected and characterized. In another example, each member is individually transformed into a cell, and the cell is characterized, e.g., using a nucleic acid microarray to determine if the transcription of endogenous genes is altered (see “Profiling Regulatory Properties of a Chimeric Zinc Finger Protein,” below).

Introducing Nucleic Acid Libraries into Cells

Library nucleic acids can be introduced into cells by a variety of methods. In one example, the library is stored as a random pool including multiple replicates of each library nucleic acid. An aliquot of the pool is transformed into cells. In another embodiment, individual library members are stored separately (e.g., in separate wells of a microtitre plate or at separate addresses of an array) and are individually introduced into cells.

In still another embodiment, the library members are stored in pools that have a reduced complexity relative to the library as a whole. For example, each pool can include 10³ different library members from a library of 10⁵ or 10⁶ different members. When a pool is identified as having a member that causes a particular effect, the pool is deconvolved to identify the individual library member that mediates the phenotypic effect. This approach is useful when recovery of the altered cell is difficult, e.g., in a screen for chimeric proteins that cause apoptosis.

Library nucleic acids can be introduced into cells by a variety of methods. Exemplary methods include electroporation (see, e.g., U.S. Pat. No. 5,384,253); microprojectile bombardment techniques (see, e.g., U.S. Pat. Nos. 5,550,318; 5,538,880; and 5,610,042; and WO 94/09699); liposome-mediated transfection (e.g., using LIPOFECTAMINE™ (Invitrogen) or SUPERFECT™ (QIAGEN GmbH); see, e.g., Nicolau et al., Methods Enzymol., 149:157-176, 1987.); calcium phosphate or DEAE-Dextran mediated transformation (see, e.g., Rippe et al., (1990) Mol. Cell Biol., 10:689-695); direct microinjection or sonication loading; receptor mediated transfection (see, e.g., EP 273 085); and Agrobacterium-mediated transformation (see, e.g., U.S. Pat. Nos. 5,563,055 and 5,591,616). The term “transform,” as used herein, encompasses any method that introduces an exogenous nucleic acid into a cell.

It is also possible to use a viral particle to deliver a library nucleic acid into a cell in vitro or in vivo. In one embodiment, viral packaging is used to deliver the library nucleic acids to cells within an organism. In another embodiment, the library nucleic acids are introduced into cells in vitro, after which the cells are transferred into an organism.

After introduction of the library nucleic acids, the library nucleic acids are expressed so that the chimeric proteins encoded by the library are produced by the cells. Constant regions of the library nucleic acid can provide necessary regulatory and supporting sequences to enable expression. Such sequences can include transcriptional promoters, transcription terminators, bacterial origins of replication, markers for indicating the presence of the library nucleic acid or for selection of the library nucleic acid.

Screening Nucleic Acid Libraries Encoding Chimeric Proteins

In a screen, the cells are evaluated to identify ones that have an altered phenotype. This process can be adapted to the phenotype of interest. As the number of possible phenotypes is vast, so too are the possibilities for screening. Numerous genetic screens and selections have been conducted to identify mutants or overexpressed naturally occurring genes that result in particular phenotypes. Any of these methods can be adapted to identify useful members of a nucleic acid library encoding chimeric proteins. A screen can include evaluating each cell that includes a library nucleic acid or a selection, e.g., evaluating cells or organisms that survive or otherwise withstand a particular treatment.

Exemplary methods for evaluating cells include microscopy (e.g., light, confocal, fluorescence, scanning electron, and transmission electron), fluorescence based cell sorting, differential centrifugation, differential binding, immunoassays, enzymatic assays, growth assays, and in vivo assays.

Some screens involve particular environmental conditions. Cells that are sensitive or resistant to the condition are identified.

Some screens require detection of a particular behavior of a cell (e.g., morphological changes). In one embodiment, the cells or organisms can be evaluated directly, e.g., by visual inspection, e.g., using a microscope and optionally computer software to automatically detect altered cells. In another embodiment, the cells or organisms can be evaluated using an assay or other indicator associated with the desired phenotype.

Some screens relate to cell growth. Cells that multiply at a different rate relative to a reference cell (e.g., a normal cell) are identified.

Changes in cell signaling pathways can be detected by the use of probes correlated with activity or inactivity of the pathway or by observable indications correlated with activity or inactivity of the pathway.

Some screens relate to production of a compound of interest, e.g., a metabolite, or a secreted protein. For example, cells can be identified that produce an increased amount of a compound. In another example, cells can be identified that produce a reduced amount of a compound, e.g., an undesired byproduct. Cells of interest can be identified by a variety of means, including the use of a responder cell, microarrays, chemical detection assays, and immunoassays.

Production of Cellular Products.

The invention features artificial polypeptides (e.g., chimeric zinc finger proteins) that alter the ability of a cell to produce a cellular product, e.g., a protein or metabolite. A cellular product can be an endogenous or heterologous molecule. For example, it is possible to identify an artificial polypeptide that increases the ability of a cell to produce proteins, e.g., particular proteins (e.g., particular endogenous proteins), overexpressed proteins, or heterologous proteins.

In one embodiment, cells are screened for their ability to produce a reporter protein, e.g., a protein that can be enzymatically or fluorescently detected. In one example, the reporter protein is insoluble when overexpressed in a reference cell. For example, bacterial cells can be screened for artificial polypeptides that reduce inclusion bodies. In another example, the reporter protein is secreted. Cells can be screened, e.g., for higher secretory through-put or proteolytic processing.

In one embodiment, cells are screened for their ability to alter (e.g., increase or decrease) the activity of two different reporter proteins. The reporter proteins may differ, e.g., by activity, localization (e.g., secreted/cytoplasmic/nuclear), size, solubility, isoelectric point, oligomeric state, post-translational regulation, translational regulation, and transcriptional regulation (e.g., the gene encoding them may be regulated by different regulatory sequences). The invention includes artificial polypeptides (e.g., zinc finger proteins) that alter at least two different reporter genes that differ by these properties, and zinc finger proteins that selectively regulate a reporter gene, or a class of reporter genes defined by one of these properties.

Because the phenotypic screening method can be used to isolate the artificial polypeptide, it is not necessary to know a priori how the zinc finger protein mediates increased protein production. Possible mechanisms, which can be verified, include alteration of one or more of the following: translation machinery, transcript processing, transcription, secretion, protein degradation, stress resistance, catalytic activity, e.g., metabolite production. In one example, an artificial polypeptide may modulate expression of one or more enzymes in a metabolic pathway and thereby enhance production of a cellular product such as a metabolite or a protein.

Iterative Design

Once a chimeric DNA binding protein is identified, its ability to alter a phenotypic trait of a cell can be further improved by a variety of strategies. Small libraries, e.g., having about 6 to 200 or 50 to 2000 members, or large libraries can be used to optimize the properties of a particular identified chimeric protein.

In a first exemplary implementation of an iterative design, mutagenesis techniques are used to alter the original chimeric DNA binding protein. The techniques are applied to construct a second library whose members include members that are variants of an original protein, for example, a protein identified from a first library. Examples of these techniques include: error-prone PCR (Leung et al. (1989) Technique 1:11-15), recombination, DNA shuffling using random cleavage (Stemmer (1994) Nature 389-391), Coco et al. (2001) Nature Biotech. 19:354, site-directed mutagenesis (Zollner et al. (1987) Nucl Acids Res 10:6487-6504), cassette mutagenesis (Reidhaar-Olson (1991) Methods Enzymol. 208:564-586) incorporation of degenerate oligonucleotides (Griffiths et al. (1994) EMBO J. 13:3245); serial ligation, pooling specific library members from a prefabricated and arrayed library, recombination (e.g., sexual PCR and “DNA Shuffling™” (Maxygen, Inc., CA)), or by combinations of these methods.

In one embodiment, a library is constructed that mutates a set of amino acid positions. For example, for a chimeric zinc finger protein, the set of amino acid positions may be positions in the vicinity of the DNA contacting residues, but not the DNA contacting residues themselves. In another embodiment, the library varies each encoded domain in a chimeric protein, but to a more limited extent than the initial library from which the chimeric DNA binding protein was identified. For a chimeric zinc finger protein, the nucleic acids that encode a particular domain can be varied among other zinc finger domains whose recognition specificity is known to be similar to that of the domain present in the original chimeric protein.

Some techniques include generating new chimeric DNA binding proteins from nucleic acids encoding domains of at least two chimeric DNA binding proteins that are known to have a particular functional property. These techniques, which include DNA shuffling and standard domain swapping, create new combinations of domains. See, e.g., U.S. Pat. No. 6,291,242. DNA shuffling can also introduce point mutations in addition to merely exchanging domains. The shuffling reaction is seeded with nucleic acid sequences encoding chimeric proteins that induces a desired phenotype. The nucleic acids are shuffled. A secondary library is produced from the shuffling products and screened for members that induce the desired phenotype, e.g., under similar or more stringent conditions. If the initial library is comprehensive such that chimeras of all possible domain combinations are screened, DNA shuffling of domains isolated from the same initial library may be of no avail. DNA shuffling may be useful in instances where coverage is comprehensive and also in instances where comprehensive screening may not be practical.

In a second exemplary implementation of an iterative design, a chimeric DNA binding protein that produces a desired phenotype is altered by varying each domain. Domains can be varied sequentially, e.g., one-by-one, or greater than one at a time.

The following example refers to an original chimeric protein that includes three zinc finger domains: fingers I, II, and III and that produces a desired phenotype. A second library is constructed such that each nucleic acid member of the second library encodes the same finger II and finger III as the initially identified protein. However, the library includes nucleic acid members whose finger I differs from finger I of the original protein. The difference may be a single nucleotide that alters the amino acid sequence of the encoded chimeric protein or may be more substantial. The second library can be constructed, e.g., such that the base-contacting residues of finger I are varied, or that the base-contacting residues of finger I are maintained but that adjacent residues are varied. The second library can also to include a large enough set of zinc finger domains to recognize at least 20, 30, 40, or 60 different trinucleotide sites.

The second library is screened to identify members that alter a phenotype of a cell or organism. The extent of alteration can be similar to that produced by the original protein or greater than that produced by the original protein.

Concurrently, or subsequently, a third library can be constructed that varies finger II, and a fourth library can be constructed that varies finger III. It may not be necessary to further improve a chimeric protein by varying all domains, if the chimeric protein or already identified variants are sufficient. In other cases, it is desirable to re-optimize each domain.

If other domains are varied concurrently, improved variants from each particular library can be recombined with each other to generate still another library. This library is similarly screened.

In a third exemplary implementation of an iterative design, the method includes adding, substituting, or deleting a domain, e.g., a zinc finger domain or a regulatory domain. An additional zinc finger domain may increase the specificity of a chimeric protein and may increase its binding affinity. In some cases, increased binding affinity may enhance the phenotype that the chimeric protein produces. An additional regulatory domain, e.g., a second activation domain or a domain that recruits an accessory factor, may also enhance the phenotype that the chimeric protein produces. A deletion may improve or broaden the specificity of the activity of the chimeric protein, depending on the contribution of the domain that is deleted, and so forth.

In a fourth exemplary implementation of an iterative design, the method includes co-expressing the original chimeric protein and a second chimeric DNA binding protein in a cell. The second chimeric protein can be also identified by screening a nucleic acid library that encodes different chimeras. In one embodiment, the second chimeric protein is identified by screening the library in a cell that expresses the original chimeric protein. In another embodiment, the second chimeric protein is identified independently.

Profiling Regulatory Properties of a Chimeric Zinc Finger Protein

A chimeric polypeptide that alters a phenotype of a cell can be further characterized to identify the endogenous genes that it directly or indirectly regulates. Typically, the chimeric polypeptide is produced within the cell. At an appropriate time, e.g., before, during, or after the phenotypic change occurs, the cell is analyzed to determine the levels of transcripts or proteins present in the cell or in the medium surrounding the cell. For example, mRNA can be harvested from the cell and analyzed using a nucleic acid microarray.

Nucleic acid microarrays can be fabricated by a variety of methods, e.g., photolithographic methods (see, e.g., U.S. Pat. No. 5,510,270), mechanical methods (e.g., directed-flow methods as described in U.S. Pat. No. 5,384,261), and pin based methods (e.g., as described in U.S. Pat. No. 5,288,514). The array is synthesized with a unique capture probe at each address, each capture probe being appropriate to detect a nucleic acid for a particular expressed gene.

Methods for isolating prokaryotic and eukaryotic RNAs are known. Isolated RNAs can be reverse-transcribed and optionally amplified, e.g., by rtPCR, e.g., as described in (U.S. Pat. No. 4,683,202). The nucleic acid can be labeled during amplification or reverse transcription, e.g., by the incorporation of a labeled nucleotide. Examples of preferred labels include fluorescent labels, e.g., red-fluorescent dye Cy5 (Amersham) or green-fluorescent dye Cy3 (Amersham). Alternatively, the nucleic acid can be labeled with biotin, and detected after hybridization with labeled streptavidin, e.g., streptavidin-phycoerythrin (Molecular Probes).

The labeled nucleic acid is then contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids are contacted to an array under hybridization conditions. The array is washed, and then imaged to detect fluorescence at each address of the array.

A general scheme for producing and evaluating profiles includes detecting hybridization at each address of the array. The extent of hybridization at an address is represented by a numerical value and stored, e.g., in a vector, a one-dimensional matrix, or one-dimensional array. The vector x has a value for each address of the array. For example, a numerical value for the extent of hybridization at a particular address is stored in variable xₐ. The numerical value can be adjusted, e.g., for local background levels, sample amount, and other variations. Nucleic acid is also prepared from a reference sample and hybridized to the same or a different array. The vector y is construct identically to vector x. The sample expression profile and the reference profile can be compared, e.g., using a mathematical equation that is a function of the two vectors. The comparison can be evaluated as a scalar value, e.g., a score representing similarity of the two profiles. Either or both vectors can be transformed by a matrix in order to add weighting values to different genes detected by the array.

The expression data can be stored in a database, e.g., a relational database such as a SQL database (e.g., Oracle or Sybase database environments). The database can have multiple tables. For example, raw expression data can be stored in one table, wherein each column corresponds to a gene being assayed, e.g., an address or an array, and each row corresponds to a sample. A separate table can store identifiers and sample information, e.g., the batch number of the array used, date, and other quality control information.

Genes that are similarly regulated can be identified by clustering expression data to identify coregulated genes. Such cluster may be indicative of a set of genes coordinately regulated by the chimeric zinc finger protein. Genes can be clustered using hierarchical clustering (see, e.g., Sokal and Michener (1958) Univ. Kans. Sci. Bull. 38:1409), Bayesian clustering, k-means clustering, and self-organizing maps (see, Tamayo et al. (1999) Proc. Natl. Acad. Sci. USA 96:2907).

The similarity of a sample expression profile to a reference expression profile (e.g., a control cell) can also be determined, e.g., by comparing the log of the expression level of the sample to the log of the predictor or reference expression value and adjusting the comparison by the weighting factor for all genes of predictive value in the profile.

Proteins can also be profiled in a cell that has an active chimeric protein with in it. One exemplary method for profiling proteins includes 2-D gel electrophoresis and mass spectroscopy to characterize individual protein species. Individual “spots” on the 2-D gel are proteolyzed and then analyzed on the mass spectrometer. This method can identify both the protein component and, in many cases, translational modifications.

The protein and nucleic acid profiling methods can not only provide information about the properties of the chimeric protein, but also information about natural mechanisms operating within the cell. For example, the proteins or nucleic acids upregulated by expression of the chimeric protein may be the natural effectors of the phenotypic change caused by expression of the chimeric protein.

In addition, other methods can be used to identify target genes and proteins that are directly or indirectly regulated by the artificial chimeric protein. In one example, alterations that compensate (e.g., suppress) the phenotypic effect of the artificial chimeric protein are characterized. These alterations include genetic alterations such as mutations in chromosomal genes and overexpression of a particular gene, as well as other alterations.

In a particular example, a chimeric ZFP is isolated that causes a growth defect or lethality when conditionally expressed in a cell, e.g., a pathogenic bacteria. Such a ZFP can be identified by transforming the cell with the ZFP libraries that include nucleic acids encoding ZFPs, expression of the nucleic acids being controlled by an inducible promoter. Transformants are cultured on non-inducible media and then replica-plated on both inducible and non-inducible plates. Colonies that grow normally on non-inducible plate, but show defective growth on inducible plate are identified as “conditional lethal” or “conditional growth defective” colonies.

(a) Identification of Target Genes using a cDNA Library

A cDNA expression library is then transformed into the “conditional lethal” or “conditional growth defective” strains described above. Transformants are plated on inducible plates. Colonies that survive, despite the presence and expression of the ZFP that causes the defect, are isolated. The nucleic acid sequences of cDNAs that complement the defect are characterized. These cDNA can be transcripts of direct or indirect target genes that are regulated by chimeric ZFP that mediates the defect.

(b) Identification of Target Genes using a Secondary ZFP Library

A second chimeric protein that suppresses the effect of the first chimeric protein is identified. The targets of the second chimeric protein (in the presence or absence of the first chimeric protein) are identified.

For example, a ZFP library is transformed into “conditional lethal” or “conditional growth defective” colonies (which include a first chimeric ZFP that causes the defect). Transformants are plated on inducible plates. Colonies that can survive by the expression of introduced ZFP are identified as “suppressed strains”. Target genes of the second ZFPs can be characterized by DNA microarray analysis. The comparative analysis can be done between four strains: 1) no ZFP; 2) the first ZFP alone; 3) the second ZFP alone; and 4) the first and second ZFP. For example, genes that are regulated in opposing directions by the first and second chimeric ZFPs are candidates for targets that mediate the growth-defective phenotype. This method can be applied to any phenotype, not just a growth defect.

(c) Co-Regulated Genes Identified by Expression Profiling Analysis

A candidate target of a chimeric ZFP can be identified by expression profiling. Subsequently, to determine if the candidate target mediates the phenotype of the chimeric ZFP, the candidate target can be independently over-expressed or inhibited (e.g., by genetic deletion). In addition, it may be possible to apply this analysis to multiple candidate targets since in at least some cases more than one candidate may need to be perturbed to cause the phenotype.

(d) Time-Course Analysis

The targets of a chimeric ZFP can be identified by a characterizing changes in gene expression with respect to time after a cell is exposed to the chimeric ZFP. For example, a gene encoding the chimeric ZFP can be attached to an inducible promoter. An exemplary inducible promoter is regulated by a small molecule such as doxycycline. The gene encoding the chimeric ZFP is introduced into cells. mRNA samples are obtained from cells at various times after induction of the inducible promoter.

Target DNA Site Identification

With respect to chimeric DNA binding proteins, a variety of methods can be used to determine the target site of a chimeric DNA binding protein that produces a phenotype of interest. Such methods can be used, alone or in combination, to find such a target site.

In one embodiment, information from expression profile is used to identify the target site recognized by a chimeric zinc finger protein. The regulatory regions of genes that are co-regulated by the chimeric zinc finger protein are compared to identify a motif that is common to all or many of the regulatory regions.

In another embodiment, biochemical means are used to determine what DNA site is bound by the chimeric zinc finger protein. For example, chromatin immuno-precipitation experiments can be used to isolate nucleic acid to which the chimeric zinc finger protein is bound. The isolated nucleic acid is PCR amplified and sequence. See, e.g., Gogus et al. (1996) Proc. Natl. Acad. Sci. USA. 93:2159-2164. The SELEX method is another exemplary method that can be used. Further, information about the binding specificity of individual zinc finger domains in the chimeric zinc finger protein can be used to predict the target site. The prediction can be validated or can be used to guide interpretation of other results (e.g., from chromatin immunoprecipitation, in silico analysis of co-regulated genes, and SELEX).

In still another embodiment, a potential target site is inferred based on information about the binding specificity of each component zinc finger. For example, the domains CSNR, RSNR, and QSNR have the following respective DNA binding specificities GAC, GAG, and GAA. The expected target site is formed by considering the domains in C terminal to N-terminal order and concatenating their recognition specificities to obtain one strand of the target site in 5′ to 3′ order.

Although in most cases, chimeric zinc finger proteins are likely to function as transcriptional regulators, it is possible that in some cases the chimeric zinc finger proteins mediate their phenotypic effect by binding to an RNA or protein target. Some naturally-occurring zinc finger proteins in fact bind to these macromolecules.

Additional Features of Zinc Finger Proteins

In addition to one, two, three, four, or more zinc finger domains, artificial polypeptides may optionally include a regulatory domain, or other features described herein. Regulatory domains include activation domains and repression domains. In bacteria, activation domain function can be emulated by a domain that recruits a wild-type RNA polymerase alpha subunit C-terminal domain or a mutant alpha subunit C-terminal domain, e.g., a C-terminal domain fused to a protein interaction domain. Bacterial activation domains include bacteriophage T4 Gp45-Gp55 complex, class II catabolite activator protein, also known as CRP, and bacteriophage Mu Mor protein (see also Hochschild and Dove, Cell. 92: 597-600, 1998). Bacterial repression domains also, in many cases, also act by binding a C-terminal domain of an RNA polymerase alpha subunit (Hochschild and Dove, Cell. 92: 597-600, 1998).

Peptide Linkers. Zinc finger domains can be connected by a variety of linkers. The utility and design of linkers are well known in the art. A particularly useful linker is a peptide linker that is encoded by nucleic acid. Thus, one can construct a synthetic gene that encodes a first DNA binding domain, the peptide linker, and a second DNA binding domain. This design can be repeated in order to construct large, synthetic, multi-domain DNA binding proteins. PCT WO 99/45132 and Kim and Pabo ((1998) Proc. Natl. Acad. Sci. USA 95:2812-7) describe the design of peptide linkers suitable for joining zinc finger domains.

Additional peptide linkers are available that form random coil, α-helical or β-pleated tertiary structures. Polypeptides that form suitable flexible linkers are well known in the art (see, e.g., Robinson and Sauer (1998) Proc Natl Acad Sci USA. 95:5929-34). Flexible linkers typically include glycine, because this amino acid, which lacks a side chain, is unique in its rotational freedom. Serine or threonine can be interspersed in the linker to increase hydrophilicity. In additional, amino acids capable of interacting with the phosphate backbone of DNA can be utilized in order to increase binding affinity. Judicious use of such amino acids allows for balancing increases in affinity with loss of sequence specificity. If a rigid extension is desirable as a linker, α-helical linkers, such as the helical linker described in Pantoliano et al. (1991) Biochem. 30:10117-10125, can be used. Linkers can also be designed by computer modeling (see, e.g., U.S. Pat. No. 4,946,778). Software for molecular modeling is commercially available (e.g., from Molecular Simulations, Inc., San Diego, Calif.). The linker is optionally optimized, e.g., to reduce antigenicity and/or to increase stability, using standard mutagenesis techniques and appropriate biophysical tests as practiced in the art of protein engineering, and functional assays as described herein.

For implementations utilizing zinc finger domains, the peptide that occurs naturally between zinc fingers can be used as a linker to join fingers together. A typical such naturally occurring linker is: Thr-Gly-(Glu or Gln)-(Lys or Arg)-Pro-(Tyr or Phe) (SEQ ID NO:124).

Dimerization Domains. An alternative method of linking DNA binding domains is the use of dimerization domains, especially heterodimerization domains (see, e.g., Pomerantz et al (1998) Biochemistry 37:965-970). In this implementation, DNA binding domains are present in separate polypeptide chains. For example, a first polypeptide encodes DNA binding domain A, linker, and domain B, while a second polypeptide encodes domain C, linker, and domain D. An artisan can select a dimerization domain from the many well-characterized dimerization domains. Domains that favor heterodimerization can be used if homodimers are not desired. A particularly adaptable dimerization domain is the coiled-coil motif, e.g., a dimeric parallel or anti-parallel coiled-coil. Coiled-coil sequences that preferentially form heterodimers are also available (Lumb and Kim, (1995) Biochemistry 34:8642-8648). Another species of dimerization domain is one in which dimerization is triggered by a small molecule or by a signaling event. For example, a dimeric form of FK506 can be used to dimerize two FK506 binding protein (FKBP) domains. Such dimerization domains can be utilized to provide additional levels of regulation.

Expression of Zinc Finger Proteins

Method described herein can include use of routine techniques in the field of molecular biology, biochemistry, classical genetics, and recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

In addition to other methods described herein, nucleic acids encoding zinc proteins can be constructed using synthetic oligonucleotides as linkers to construct a synthetic gene. In another example, synthetic oligonucleotides are used and/or primers to amplify sequences encoding one or more zinc finger domains, e.g., from an RNA or DNA template, artificial or synthetic. See U.S. Pat. Nos. 4,683,195 and 4,683,202; PCR Protocols: A Guide to Methods and Applications (Innis et al., eds, 1990)). Methods such as polymerase chain reaction (PCR) can be used to amplify nucleic acid sequences directly from mRNA, from cDNA, from genomic, cDNA, or zinc finger protein libraries. Degenerate oligonucleotides can be designed to amplify homologs using the sequences provided herein. Restriction endonuclease sites can be incorporated into the primers.

Gene expression of zinc finger proteins can also be analyzed by techniques known in the art, e.g., reverse transcription and amplification of mRNA, isolation of total RNA or polyA⁺ RNA, northern blotting, dot blotting, in situ hybridization, RNase protection, nucleic acid array technology, e.g., and the like.

The polynucleotide encoding an artificial zinc finger protein can be cloned into vectors before transformation into prokaryotic or eukaryotic cells for replication and/or expression. These vectors are typically prokaryote vectors, e.g., plasmids, phage or shuttle vectors, or eukaryotic vectors.

Protein Expression. To obtain recombinant expression (e.g., high level) expression of a polynucleotide encoding an artificial zinc finger protein, one can subclone the relevant coding nucleic acids into an expression vector that contains a strong promoter to direct transcription, a transcription/translation terminator, and a ribosome binding site for translational initiation. Suitable bacterial promoters are well known in the art and described, e.g., in Sambrook et al., and Ausubel et al, supra. Bacterial expression systems for expression are available in, e.g., E. coli, Bacillus sp., and Salmonella (Palva et al., (1983) Gene 22:229-235; Mosbach et al., (1983) Nature 302:543-545. Kits for such expression systems are commercially available. Eukaryotic expression systems for mammalian cells, yeast (e.g., S. cerevisiae, S. pombe, Pichia, and Hanseula), and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

A nucleic acid sequence encoding a chimeric zinc finger protein can be cloned into a vector that will permit regulatable expression of the artificial polypeptide, e.g., an inducible expression vector as described in Kang and Kim, (2000) J Biol Chem 275:8742. The inducible expression vector can include a regulatable promoter or regulatory sequence. A useful promoter or sequence for controlling expression of an artificial polypeptide is one that is selectively activated or repressed in certain conditions. Regulatable promoters include promoters responsive to an environmental parameter, e.g., thermal changes, hormones, metals, metabolites, antibiotics, or chemical agents. By modulating the concentration of an agent that can regulate the promoter or sequence, the expression of the target prokaryotic gene (e.g., the endogenous gene) can be regulated in a concentration dependent manner.

Regulatable promoters appropriate for use in E. coli include promoters which contain transcription factor binding sites from the lac, tac, trp, trc, and tet operator sequences, or operons, the alkaline phosphatase promoter (pho), an arabinose promoter such as an araBAD promoter, the rhamnose promoter, the promoters themselves, or functional fragments thereof (see, e.g., Elvin et al., 1990, Gene 37: 123-126; Tabor and Richardson, 1998, Proc. Natl. Acad. Sci. U.S.A. 1074-1078; Chang et al., 1986, Gene 44: 121-125; Lutz and Bujard, March 1997, Nucl. Acids. Res. 25: 1203-1210; D. V. Goeddel et al., Proc. Nat. Acad. Sci. U.S.A., 76:106-110, 1979; J. D. Windass et al. Nucl. Acids. Res., 10:6639-57, 1982; R. Crowl et al., Gene, 38:31-38, 1985; Brosius, 1984, Gene 27: 161-172; Amanna and Brosius, 1985, Gene 40: 183-190; Guzman et al., 1992, J. Bacteriol., 174: 7716-7728; Haldimann et al., 1998, J. Bacteriol., 180: 1277-1286). Inducible promoter systems such as lac promoters may be bound by repressor or inducer molecules. Lac promoters are induced by lactose or structurally related molecules such as isopropyl-beta-D-thiogalactoside (IPTG) and are repressed by glucose. Some inducible promoters are induced by a process of derepression, e.g., inactivation of a repressor molecule.

A regulatable promoter sequence can also be indirectly regulated. Examples of promoters that can be engineered for indirect regulation include: the phage lambda PR, —PL, phage T7, SP6, and T5 promoters. For example, the regulatory sequence is repressed or activated by a factor whose expression is regulated, e.g., by an environmental parameter. One example of such a promoter is a T7 promoter. The expression of the T7 RNA polymerase can be regulated by an environmentally-responsive promoter such as the lac promoter. For example, the cell can include an artificial nucleic acid that includes a sequence encoding the T7 RNA polymerase and a regulatory sequence (e.g., the lac promoter) that is regulated by an environmental parameter (Studier, F. W., and Moffatt, B. A. J. Mol. Biol. 189(1): 113-30, 1986). The activity of the T7 RNA polymerase can also be regulated by the presence of a natural inhibitor of RNA polymerase, such as T7 lysozyme (Studier, F. W. J. Mol. Biol. 219(1): 37-44, 1991).

In addition to the promoter, the expression vector typically contains a transcription unit or expression cassette that contains all the additional elements required for expression in host cells. A typical expression cassette thus contains a promoter operably linked to the coding nucleic acid sequence and signals appropriate for efficient expression in the host cell type, e.g., polyadenylation of the transcript, ribosome binding sites, and translation termination. Additional elements of the cassette, e.g., for expression in eukaryotes, may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc-, or a hexa-histidine tag.

Expression vectors can contain regulatory elements from eukaryotic viruses, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal. Inducible expression vectors are often chosen if expression of the protein of interest is detrimental to eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with mitochondrial respiratory chain protein encoding sequences and glycolysis protein encoding sequence under the direction of the polyhedrin promoter or other strong baculovirus promoters

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The prokaryotic sequences can be chosen such that they do not interfere with the replication of the DNA in eukaryotic cells.

Standard transfection methods are used to produce bacterial, mammalian, yeast or insect cell lines that express large quantities of zinc finger proteins, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra).

After the expression vector is introduced into the cells, the transfected cells are cultured under conditions favoring expression or activating expression. The protein can then be isolated from a cell extract, cell membrane component or vesicle, or media.

Expression vectors with appropriate regulatory sequences can also be used to express a heterologous gene encoding an artificial zinc finger in a model organism, e.g., a Drosophila, nematode, zebrafish, Xenopus, or mouse. See, e.g., Riddle et al., eds., C. elegans II. Plainview (NY): Cold Spring Harbor Laboratory Press; 1997.

Protein Purification. Zinc finger protein can be purified from materials generated by any suitable expression system, e.g., those described above.

Zinc finger proteins may be purified to substantial purity by standard techniques, including selective precipitation with such substances as ammonium sulfate; column chromatography, affinity purification, immunopurification methods, and others (see, e.g., Scopes, Protein Purification: Principles and Practice (1982); U.S. Pat. No. 4,673,641; Ausubel et al., supra; and Sambrook et al., supra). For example, zinc finger proteins can include an affinity tag that can be used for purification, e.g., in combination with other steps.

Recombinant proteins are expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein. Proteins expressed in bacteria may form insoluble aggregates (“inclusion bodies”). Several protocols are suitable for purifying proteins from inclusion bodies. See, e.g., Sambrook et al., supra; Ausubel et al., supra). If the proteins are soluble or exported to the periplasm, they can be obtained from cell lysates or periplasmic preparations.

Differential Precipitation. Salting-in or out can be used to selectively precipitate a zinc finger protein or a contaminating protein. An exemplary salt is ammonium sulfate. Ammonium sulfate precipitates proteins on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration precipitates many of the more hydrophobic proteins. The precipitate is analyzed to determine if the protein of interest is precipitated or in the supernatant. Ammonium sulfate is added to the supernatant to a concentration known to precipitate the protein of interest. The precipitate is then solubilized in buffer and the excess salt removed if necessary, either through dialysis or diafiltration.

Column chromatography. A zinc finger protein can be separated from other proteins on the basis of its size, net surface charge, hydrophobicity, and affinity for ligands. In addition, antibodies raised against proteins can be conjugated to column matrices and the proteins immunopurified. All of these methods are well known in the art. Chromatographic techniques can be performed at any scale and using equipment from many different manufacturers (e.g., Pharmacia Biotech). See, generally, Scopes, Protein Purfication: Principles and Practice (1982).

Similarly general protein purification procedures can be used to recover a protein whose production is altered (e.g., enhanced) by expression of an artificial zinc finger protein in a producing cell.

The invention also provides compositions, e.g., pharmaceutically acceptable compositions, which include an artificial polypeptide, e.g., as described herein, or a nucleic acid encoding such a factor formulated together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions may be in a variety of forms. These include, for example, liquid, semi-solid and solid dosage forms, such as liquid solutions (e.g., injectable and infusible solutions), dispersions or suspensions, tablets, pills, powders, and liposomes.

The compositions can be administered by a variety of methods known in the art, although for many applications, the route/mode of administration is intravenous injection or infusion. For example, the composition can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. Many methods for the preparation of such formulations are patented or generally known. See, e.g., Sustained and Controlled Release Drug Delivery Systems, J. R. Robinson, ed., Marcel Dekker, Inc., New York, 1978.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of the protein or nucleic acid is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Cell-Based Therapeutics

Cell based-therapeutic methods include introducing a nucleic acid that encoding the artificial zinc finger protein operably linked to a promoter into a cell. The artificial zinc finger protein can be selected to regulate an endogenous gene in the culture cell or to produce a desired phenotype in the cultured cell. Further, it is also possible to modify cells using nucleic acid recombination, to insert a gene encoding an artificial zinc finger protein that regulates an endogenous gene. The cell can be administered to a subject.

In vivo administration generally can include administering a pharmaceutical composition containing a therapeutically-effective amount of the modified bacteria. The therapeutically effective amount will depend on the mode of administration and the strain of bacteria used. Generally, the therapeutically effective amount is an amount of bacteria sufficient to induce a desired response. In one embodiment, a given number of bacterial cells is administered. Bacteria can be administered as a function of the number of colony forming units (CFU) of the strain. For example, between 1×10³ and 1×10¹¹ CFU of bacteria can be administered per dose.

In one embodiment, bacteria are administered orally. See, e.g., Angelakopoulos H, et al. Infect Immun. 70(7): 3592-601 (2002). Briefly, bacteria are cultured, pelleted by centrifugation and washed twice with normal saline. The bacteria are resuspended at a specific turbidity for adminstration in normal saline or a solution that can buffer against gastric acid (e.g., citrate buffer (pH 7.0) containing sucrose; bicarbonate buffer (pH 7.0) alone (Levine et al, J. Clin. Invest., 79:888-902 (1987); and Black et al J. Infect. Dis., 155:1260-1265 (1987)), or bicarbonate buffer (pH 7.0) containing ascorbic acid, lactose, and optionally aspartame (Levine et al, Lancet, II:467-470 (1988)). Alternatively, a buffer solution is ingested prior to ingestion of the bacteria. The bacteria can be formulated into a pharmaceutical composition by combination with an appropriate pharmaceutically acceptable carrier. Appropriate carriers include proteins, e.g., as found in skim milk, sugars, e.g., sucrose, or polyvinylpyrrolidone. Typically these carriers can be used at a concentration of about 0.1-90% (w/v), and preferably at a range of 1-10% (w/v). The bacteria can be used alone or in appropriate association, as well as in combination with other pharmaceutically active compounds. The bacteria can be administered in combination with an adjuvant. The bacteria can be formulated into preparations in solid, semisolid, or liquid form such as tablets, capsules, powders, granules, ointments, solutions, suppositories, and injections, in usual ways for topical, nasal, oral, parenteral, or surgical administration. Administration in vivo can be oral, mucosal nasal, bronchial, parenteral, subcutaneous, intravenous, intra-arterial, intramuscular, intra-organ, intra-tumoral, or surgical. Administration can include the use of an implantable container (e.g., a biodegradable or semipermeable shell, capsule, tube or other device for delivery of the bacteria) that may optionally contain a matrix upon or into which cells may be seeded. The route of administration can be selected as is appropriate for the targeted host cells. Target cells can also be removed from the subject, treated ex vivo, and the cells then returned to the subject. Other exemplary methods for in vivo administration are described in Shen et al., Proc Natl Acad Sci USA 92(9):3987-3991, 1995; Jensen et al, Immunol Rev 158: 147-157, 1997; Szalay et al., Proc Natl Acad Sci USA 92(26):12389-12392, 1995; Belyi et al, FEMS Immunol Med Microbiol 13(3): 211-213, 1996; Frankel et al., J. Immunol 155(10):4775-4782, 1995; Goossens et al., Int Immunol 7(5):797-805, 1995; Schafer et al., J. Immunol 149(1):53-59, 1992; and Linde et al., Vaccine 9(2): 101-105, 1991.

Target for Altered Protein Production

In one embodiment, a nucleic acid library is screened to identify an artificial zinc finger protein that alters production, synthesis or activity of one or more particular target proteins in a prokaryotic cell. The alteration can increase or decrease activity or abundance of the target protein. The phenotype screened for can be associated with altered production or activity of one or more target proteins or can be the level of production or activity itself. For example, it is possible to screen a nucleic acid library for artificial polypeptides that activate or suppress expression of a reporter gene (such as those encoding luciferase, LacZ, or GFP) under the control of a regulatory sequence (e.g., the promoter) of an endogenous target gene.

The methods and compositions described herein can be applied to screening any target gene or phenotype of interest. For example, bacterial cells can be screened for a given enzyme activity. Cells having an increased or decreased amount of an enzyme activity may be isolated. Bacterial enzymes for which overexpression may be desired include oxidoreductases, transferases, hydrolases, lyases, isomerases, and ligases. Expression of zinc finger proteins may coordinately modulate expression of multiple genes, either due to the organization of prokaryotic genes in operons, or by virtue of binding to multiple independent sites. Accordingly, the methods may provide for complex effects on expression of multiple genes.

The present invention will be described in more detail through the following examples. However, it should be noted that these examples are not intended to limit the scope of the present invention.

### EXAMPLE 1

### Construction of ZFP Libraries

In one example, various phenotypes of E. coli are altered by regulating gene expression using zinc finger protein (ZFP) expression libraries. The zinc finger proteins in these exemplary libraries consist of three or four zinc finger domains (ZFDs) and recognize 9- to 12-bp DNA sequences respectively. The chimeric zinc finger protein is identified without a priori knowledge of the target genes. We used 25 different zinc finger domains as modular building blocks to construct proteins containing 3-finger or 4-finger zinc finger proteins. These libraries of ZFP expression plasmids were then transformed into E. coli. In each transformed cell, a different ZFP polypeptide is expressed and can be assayed for regulation of unspecified target genes in the genome. This alteration of gene expression pattern can lead to phenotypic changes. In addition, the regulated target genes can be identified by combining in silico prediction of target DNA sequences with genomic DNA immunoprecipitation after identifying zinc finger proteins introduced to the transformants.

(1) E. coli Strain and Plasmids

The E. coli strain used for screening of various phenotypic changes was DH5α. Strain DY330 (W3110 DlacU169 gal490 lc1857 D (cro-bioA)) was used for gene disruption by homologous recombination (Yu et al., Proc Natl Acad Sci USA. 97(11):5978-83, 2000). The parental vector to construct libraries of zinc finger protein was plasmid p3. The plasmid vector used for the expression of zinc finger protein in E. coli was pZL1,

(2) Construction of Plasmid p3

The parental vector that we used to construct libraries of zinc finger proteins is the plasmid p3. p3 was constructed by modifying the pcDNA3 vector (Invitrogen, San Diego Calif.) as follows. The pcDNA3 vector was digested with HindIII and XhoI. A synthetic oligonucleotide duplex with compatible overhangs was ligated into the digested pcDNA3. The duplex contains nucleic acid that encodes the hemagglutinin (HA) tag and a nuclear localization signal. The duplex also includes: restriction sites for BamHI, EcORI, NotI, and BglII; and a stop codon. The XmaI site in SV40 origin of the vector was destroyed by digestion with XmaI, filling in the overhanging ends of the digested XmaI restriction site, and religation of the ends.

(3) Construction of pZL1

We used pZL1 as the parental vector for conditional expression of zinc finger proteins in E. coli. PZL1 was modified from pBT-LGF2 (Clontech) to have V5 epitope and multiple cloning sites. The following nucleic acid sequences were inserted into ClaI and NotI sites of pBT-LGF2 to generate pZL1 plasmid.

| | |
|---|---|
| ATC GAT AAG CTA ATT CTC ACT CAT TAG GCA CCC CAG GCT TTA CAC | (SEQ ID NO:124) |
| TTT ATG CTT CCG GCT CGT ATA ATG TGT GGA ATT GTG AGC GGA TAA CAA | |
| TTT CAC ACA GGA AAC AGC GTC CAT GGG TAA GCC TAT CCC TAA CCC TCT | |
| CCT CGG TCT CGA TTC TAC ACA AGC TAT GGG TGC TCC TCC AAA AAA GAA | |
| GAG AAA GGT AGC TGG ATC CAC TAG TAA CGG CCG CCA GTG TGC TGG AAT | |
| TCT GCA GAT ATC CAT CAC ACT GGC GGC CGC | |

The library constructed in p3 was subcloned into into EcOR1 and NotI sites of pZL1 to generate ZFP libraries functioning in E. coli.

(4) Library construction

A three-fingered (the “3-F library”) or a four-fingered protein library (the “4-F library”) were constructed from nucleic acids encoding 25 different ZFDs (Table 4, below).

TABLE 4

Zinc finger domains for construction of 3-finger or
4-finger ZFP libraries

| Domain | | | | |
|---|---|---|---|---|
| Name | Source | Target Sites | Amino acid sequences | SEQ ID NO: |
| DSAR | Mutated¹ | GTC | FMCTWSYCGKRFTDRSALARHKRTH | 125 |
| CSNR1 | Human | GAA > GAC > GAG | YKCKQCGKAFGCPSNLRRHGRTH | 126 |
| DSCR | Human | GCC | YTCSDCGKAFRDKSCLNRHRRTH | 127 |
| DSNR | Mutated² | GAC | YACPVESCDRRFSDSSNLTRHIRIH | 128 |
| HSSR | Human | GTT | FKCPVCGKAFRHSSSLVRHQRTH | 129 |
| ISNR | Human | GAA > GAT > GAC | YRCKYCDRSFSISSNLQRHVRNIH | 130 |
| QFNR | Human | GAG | YKCHQCGKAFIQSFNLRRHERTH | 131 |
| QNTQ | Drosophila³ | ATA | YTCSYCGKSFTQSNTLKQHTRIH | 132 |
| QSHV | Human | CGA > AGA > TGA | YECDHCGKSFSQSSHLNVHKRTH | 133 |
| QSNI | Human | AAA, CAA | YMCSECGRGFSQKSNLIIHQRTH | 134 |
| QSNK | Human | GAA > TAA > AAA | YKCEECGKAFTQSSNLTKHKKIH | 135 |
| QSNR1 | Human | GAA | FECKDCGKAFIQKSNLIRHQRTH | 136 |
| QSNV2 | Human | AAA, CAA | YVCSKCGKAFTQSSNLTVHQKIH | 137 |
| QSSR1 | Human | GTA > GCA | YKCPDCGKSFSQSSSLIRHQRTH | 138 |
| QTHQ | Human | CGA > TGA, AGA | YECHDCGKSFRQSTHLTQHRRIH | 139 |
| QTHR1 | Human | GGA > AGA, GAA > TGA, CGA | YECHDCGKSFRQSTHLTRHRRIH | 140 |
| RDHT | Human | TGG, AGG, CGG, GGG | FQCKTCQRKFSRSDHLKTHTRTH | 141 |
| RDKR | Human | GGG > AGG | YVCDVEGCTWKFARSDKLNRHKKRH | 142 |
| RDNQm | Mutated⁴ | AAG | FACPECPKRFMRSDNLTQHIKTH | 143 |
| RSHR | Human | GGG | YKCMECGKAFNRRSHLTRHQRIH | 144 |
| RSNR | Human | GAG > GTG | YICRKCGRGFSRKSNLIRHQRTH | 145 |
| VSNV | Human | AAT > CAT > TAT | YECDHCGKAFSVSSNLNVHRRIH | 146 |
| VSSR | Human | GTT > GCT > GTG > GTA | YTCKQCGKAFSVSSSLRRHETTH | 147 |
| VSTR | Human | GCT > GCG | YECNYCGKTFSVSSTLIRHQRIH | 148 |
| WSNR | Human | GGT | YRCEECGKAFRWPSNLTRHKRIH | 149 |

Superscripts in column 2 of Table 4 refer to 1) Zhang et al., (2000) J. Biol. Chem. 275:33850-33860; 2) Rebar and Pabo (1994) Science 263:671-673; 3) Gogus et al., (1996) Proc. Natl. Acad. Sci. USA. 93:2159-2164; 4) Liu et al. (2001) J. Biol. Chem. 276(14):11323-11334. The small letter m after the name of certain zinc finger domains indicates that the domain obtained by mutation of a parental domain.

Nucleic acid fragments encoding each ZFD were individually cloned into the p3 vector to form “single fingered” vectors. Equal amounts of each “single fingered” vector were combined to form a pool. One aliquot of the pool was digested with AgeI and XhoI to obtain digested vector fragments. These vector fragments were treated with phosphatase for 30 minutes. Another aliquot of the pool was digested with XmaI and XhoI to obtain segments encoding single fingers. The digested vector nucleic acids from the AgeI and XhoI digested pool were ligated to the nucleic acid segments released from the vector by the XmaI and XhoI digestion. The ligation generated vectors that each encode two zinc finger domains. After transformation into E. coli, approximately 1.4×10⁴ independent transformants were obtained, thereby forming a two-fingered library. The size of the insert region of the two-fingered library was verified by PCR analysis of 40 colonies. The correct size insert was present in 95% of the library members.

To prepare a three-fingered library, DNA segments encoding one finger were inserted into plasmids encoding two fingers. The 2-fingered library was digested with AgeI and XhoI. The digested plasmids, which retain nucleic acid sequences encoding two zinc finger domains, were ligated to the pool of nucleic acid segments encoding a single finger (prepared as described above by digestion with XmaI and XhoI). The products of this ligation were transformed into E. coli to obtain about 2.4×10⁵ independent transformants. Verification of the insert region confirmed that library members predominantly included sequences encoding three zinc finger domains.

To prepare a four-fingered library, DNA segments encoding two fingers were inserted into plasmids encoding two fingers. The two-fingered library was digested with XmaI and XhoI to obtain nucleic acid segments that encode two zinc finger domains. The two-fingered library was also digested with AgeI and XhoI to obtain a pool of digested plasmids. The digested plasmids, which retain nucleic acid sequences encoding two zinc finger domains, were ligated to the nucleic acid segments encoding two zinc finger domains to produce a population of plasmids encoding different combination of four fingered proteins. The products of this ligation were transformed into E. coli and yielded about 7×10⁶ independent transformants.

3F- or 4F-ZFP inserts were subcloned into EcOR1 and NotI sites of pZL1 vector to generate ZFP libraries functioning in E. coli.

### EXAMPLE 2

### Solvent Tolerant Bacterial Cells

We screened for bacterial cells that express artificial chimeric zinc finger proteins for cells that were resistant to an organic solvent as a result of the artificial chimeric zinc finger protein. The E. coli strain DH5α was transformed with the 3-finger or 4-finger ZFP nucleic acid library formatted for prokaryotic expression. Transformants were cultured overnight in LB with chloramphenicol (34 μg/ml). The overnight-culture was diluted to 1:500 in 1 ml fresh LB media with 1 mM IPTG and chloramphenicol to induce ZFP expression. After a three hour incubation at 30° C., hexane was added to 1.5% and rapidly vortexed to make emulsion of hexane and E. coli culture. The mixture was incubated for three hours with shaking (250 rpm) at 37° C. and plated on LB plates with chloramphenicol [g/ml (34 μg/ml). Plasmids were purified from the pool of growing colonies and transformed into DH5α The transformants were treated with hexane as described above. Selection for hexane tolerance was repeated two additional times. Plasmids were recovered from 20 individual colonies that could grow on LB plates with chloramphenicol (34 μg/ml) after the third round of selection. These plasmids were retransformed into DH5α. Each transformant was retested for hexane-tolerance as described above. Plasmids that induce hexane tolerance were sequenced to characterize the encoded zinc finger protein.

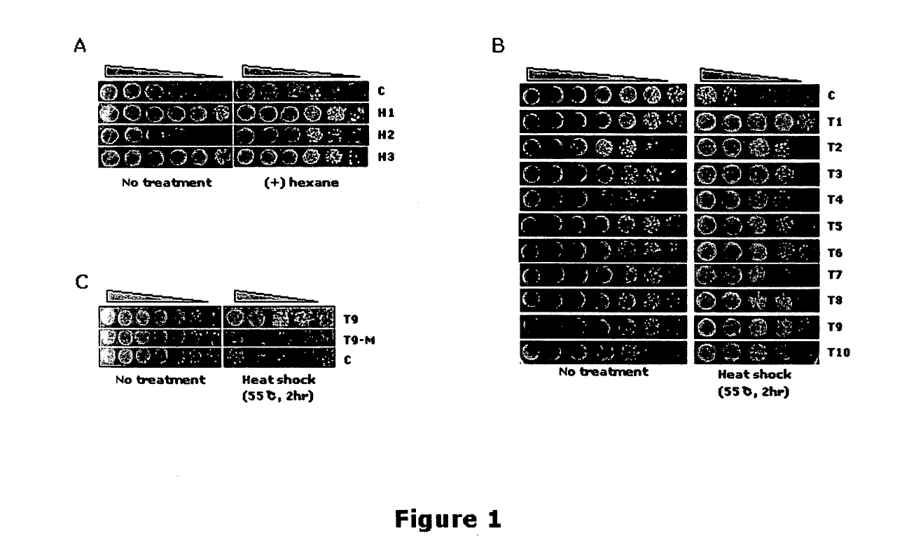

Three different zinc finger proteins were identified for their ability to confer hexane tolerance to E. coli cells. The amino acid sequences of each of these zinc finger proteins is depicted in Table 7. The sequence of each zinc finger domain of these proteins are listed in Table 1, rows 2-11. The finger motif sequences are depicted in Table 6. Hexane tolerance was evaluated by comparing the survival rate of transformants expressing one of the zinc finger proteins—H1, H2, and H3—to the survival rate control cells. The control cells either included an empty vector (C1) or ZFP-1. The ZFP-1 construct encodes a zinc finger protein that does not confer hexane resistance and that includes the fingers RDER-QSSR-DSKR. Bacterial cells that express hexane resistance-conferring zinc finger proteins exhibited as much as a 200-fold increase in hexane tolerance (Table 5, FIG. 1A).

TABLE 5
Hexane Resistant Zinc Finger Proteins.

| Expression Construct | Name | Survival Rate |
|---|---|---|
| Control | C1 | 0.14% |
| Control | ZFP-1 | 0.05% |
| Hexane resistance ZFP | H1 | 21.4% |
| Hexane resistance ZFP | H2 | 1.85% |
| Hexane resistance ZFP | H3 | 28.6 |

TABLE 6

Zinc finger motif sequences and DNA target sequences of
proteins that confer hexane tolerance in E. coli

| | | | | | | |
|---|---|---|---|---|---|---|
| | | | | | | No. of |
| Name | F1 | F2 | F3 | F4 | putative DNA target | occurrences(##) |

| | | | | | | |
|---|---|---|---|---|---|---|
| H1 | RSHR | HSSR | ISNR | | GAH GTT GGG | 5 |
| H2 | QNTQ | CSNR | ISNR | | GAH GAV ATA | 1 |
| H3 | ISNR | RDHT | QTHR1 | VSTR | GCT GRA NGG GAH | 3 |
| | | | | | (SEQ ID NO: | |

(##)Occurrence of the ZFP in nine colonies that could grow after third round of hexane tolerant screening

TABLE 7

Amino acid sequences of ZFP-TFs isolated from
E. coli phenotype screening

| | | |
|---|---|---|
| ZFP | Amino acid Sequence | SEQ ID NO: |

| | | |
|---|---|---|
| H1 | YKCMECGKAFNRRSHLTRHQRIHTGEKPFKCPVCGKAFRHSSSLVRHQRT | 51 |
| | HTGEKPYRCK YCDRSFSISS NLQRHVRNIH | |
| H2 | YTCSYCGKSFTQSNTLKQHTRIHTGEKPYKCKQCGKAFGCPSNLRRHGRT | 52 |
| | HTGEKPYRCKYCDRSFSISS NLQRHVRNIH | |
| H3 | YRCKYCDRSFSISSNLQRHVRNIHTGEKPF QCKTCQRKFS RSDHLKTHTR | 53 |
| | THTGEKPYECHDCGKSFRQSTHLTRHRRIH TGEKPYECNY CGKTFSVSST | |
| | LIRHQRIH | |

### EXAMPLE 3

### Thermo-Tolerant Bacterial Cells

We screened for zinc finger proteins that conferred heat resistance to cells. The nucleic acid library encoding different zinc finger proteins was transformed into E. coli cellsand cultured overnight in LB with chloramphenicol (34 μg/ml). The overnight-culture was diluted to 1:500 in 1 ml fresh LB media with 1 μM IPTG and chloramphenicol (34 μg/ml) to induce ZFP expression. After a 3 hour incubation at 30° C., 100 ul culture was transferred to micro-centrifuge tube and incubated in water bath at 55° C. for 2 hrs. The culture was plated on LB plate with chloramphenicol (34 μg/ml). Plasmids were purified from the pool of growing colonies and transformed into DH5α. Selection for thermotolerance was repeated with retransformants. Plasmid was purified from 30 individual colonies that could grow on LB+ chloramphenicol plate (34 μg/ml) after third round of selection and retransformed into DH5α Each transformant was analyzed for thermo-tolerance as described above. Plasmids that could induce thermo-tolerance were sequenced to identify ZFP.

Ten different zinc finger proteins were identified and the improvement of thermo-tolerance was analyzed by comparing survival rate of ZFP transformants and control cells, C1 or ZFP-2 upon heat treatment. The amino acid sequences of each of these zinc finger proteins is depicted in Table 9. The sequence of each zinc finger domain of these proteins are listed in Table 1, rows 12-51. The finger motif sequences are depicted in Table 8. C1 or ZFP-2 represent the transformants of empty vector or a control ZFP that has no effect on thermotolerance (QTHQ-RSHR-QTHR1), respectively. More than 99.99% of wild type cells died upon heat treatment at 55° C. for 2 hours. In contrast, about 6% of cells transformed with certain ZFP-TFs survived under these extreme conditions, a 700 fold increase in the thermotolerance phenotype—that is, the percentage of cells expressing ZFP-TFs that survive under stress conditions (6.3%) divided by the percentage of C1 that survived under the same conditions (0.0085%) (FIG. 1B).

TABLE 8
ZFPs that confer thermotolerance.

| | | | | | | |
|---|---|---|---|---|---|---|
| Name | F1 | F2 | F3 | F4 | putative DNA target | occurrences |

| | | | | | | |
|---|---|---|---|---|---|---|
| T-1 | QSHV | VSNV | QSNK | QSNK | 5′DAA DAA AAT HGA 3′ | 6 |
| | | | | | (SEQ ID NO:150) | |
| T-2 | RDHT | QSHV | QTHR1 | QSSR1 | 5′GYA GRA HGA NGG K 3′ | 3 |
| | | | | | (SEQ ID NO:151) | |
| T-3 | WSNR | QSHV | VSNV | QSHV | 5′HGA AAT HGA GGT 3′ | 1 |
| | | | | | (SEQ ID NO:152) | |
| T-4 | QTHR1 | RSHR | QTHR1 | QTHR1 | 5′GRA GRA GGG GRA 3′ | 1 |
| | | | | | (SEQ ID NO:153) | |
| T-5 | DSAR | RDHT | QSHV | QTHR1 | 5′GRA HGA NGG GTC 3′ | 2 |
| | | | | | (SEQ ID NO:154) | |
| T-6 | QTHQ | RSHR | QTHR1 | QTHR1 | 5′GRA GRA GGG HGA 3′ | 1 |
| | | | | | (SEQ ID NO:155) | |
| T-7 | QSHV | VSNV | QSNR1 | CSNR1 | 5′GAV GAA AAT HGA 3′ | 3 |
| | | | | | (SEQ ID NO:156) | |
| T-8 | VSNV | QTHR1 | QSSR1 | RDHT | 5′NGG GYA GRA AAT 3′ | 2 |
| | | | | | (SEQ ID NO:157) | |
| T-9 | RDHT | QSHV | QTHR1 | QSNR1 | 5′GAA GRA HGA NGG K 3′ | 2 |
| | | | | | (SEQ ID NO:158) | |
| T-10 | DSAR | RDHT | QSNK | QTHR1 | 5′GRA DAA NGG GTC 3′ | 2 |
| | | | | | (SEQ ID NO:159) | |

TABLE 9

Amino acid sequences of ZFP-TFs isolated from
E. coli phenotype screening

| | | |
|---|---|---|
| ZFP | Amino acid | SEQ ID NO: |

| | | |
|---|---|---|
| T1 | YECDHCGKSF SQSSHLNVHK RTHTGEKPYE CDHCGKAFSV | 54 |
| | SSNLNVHRRI HTGEKPYKCE ECGKAFTQSS NLTKHKKIHT | |
| | GEKPYKCEEC GKAFTQSSNL TKHKKIH | |
| T2 | FQCKTCQRKF SRSDHLKTHT RTHTGEKPYE CDHCGKSFSQ | 55 |
| | SSHLNVHKRT HTGEKPYECH DCGKSFRQST HLTRHRRIHT | |
| | GEKPYKCPDC GKSFSQSSSL IRHQRTH | |
| T3 | YRCEECGKAF RWPSNLTRHK RIHTGEKPYE CDHCGKSFSQ | 56 |
| | SSHLNVHKRT HTGEKPYECD HCGKAFSVSS NLNVHRRIHT | |
| | GEKPYECDHC GKSFSQSSHL NVHKRTH | |
| T4 | YECHDCGKSF RQSTHLTRHR RIHTGEKPYK CMECGKAFNR | 57 |
| | RSHLTRHQRI HTGEKPYECH DCGKSFRQST HLTRHRRIHT | |
| | GEKPYECHDC GKSFRQSTHL TRHRRIH | |
| T5 | FMCTWSYCGK RFTDRSALAR HKRTHTGEKP FQCKTCQRKF | 58 |
| | SRSDHLKTHT RTHTGEKPYE CDHCGKSFSQ SSHLNVHKRT | |
| | HTGEKPYECH DCGKSFRQST HLTRHRRIH | |
| T6 | YECHDCGKSF RQSTHLTQHR RIHTGEKPYK CMECGKAFNR | 59 |
| | RSHLTRHQRI HTGEKPYECH DCGKSFRQST HLTRHRRIHT | |
| | GEKPYECHDC GKSFRQSTHL TRHRRIH | |
| T7 | YECDHGGKSF SQSSHLNVHK RTHTGEKPYE CDHCGKAFSV | 60 |
| | SSNLNVHRRI HTGEKPFECK DCGKAFIQKS NLIRHQRTHT | |
| | GEKPYKCKQC GKAFGCPSNL RRHGRTH | |
| T8 | YECDHCGKAF SVSSNLNVHR RIHTGEKPYE CHDCGKSFRQ | 61 |
| | STHLTRHRRI HTGEKPYKCP DCGKSFSQSS SLIRHQRTHT | |
| | GEKPFQCKTC QRKFSRSDHL KTHTRTH | |
| T9 | FQCKTCQRKF SRSDHLKTHT RTHTGEKPYE CDHCGKSFSQ | 62 |
| | SSHLNVHKRT HTGEKPYECH DCGKSFRQST HLTRHRRIHT | |
| | GEKPFECKDC GKAFIQKSNL IRHQRTH | |
| T10 | FMCTWSYCGK RFTDRSALAR HKRTHTGEKP FQCKTCQRKF | 63 |
| | SRSDHLKTHT RTHTGEKPYK CEECGKAFTQ SSNLTKHKKI | |
| | HTGEKPYECH DCGKSFRQST HLTRHRRIH | |

The T9 ZFP was further analyzed by site-directed mutagenesis of an arginine residue critical for DNA binding to an alanine. The mutated T9 ZFP (T9-M) failed to induce heat shock resistance in E. coli (FIG. 1C), suggesting that the capability of T9 ZFP-TF to induce thermotolerance is dependent on the binding of ZFP to the target DNA.

### EXAMPLE 4

### Identification of ZFP Target Genes

A benefit of the ZFP approach, in contrast to chemical or UV mutagenesis, is that it allows for the identification and characterization of target gene associated with the improved phenotype based on the expected binding sequences of ZFP.

A combined approach of chromatin immuno-precipitation and in silico prediction of binding sites of ZFP was undertaken to identify target genes of T9 ZFP that induce thermo-tolerance in E. coli. E. coli genomic DNA fragments that were cross-linked with T9 ZFP were immuno-precipitated by the modified chromatin immuno-precipitation method (Weinmann & Farnham, Methods. 26(1):37-47, 2002).

Briefly, E. coli cells were grown to an OD₆₀₀ of 1.0˜1.5 in 100 ml LB medium containing chloramphenicol and 1 mM IPTG. Formaldehyde was added at a final concentration of 1% directly to medium. Fixation proceeded at room temperature with gentle swirling for 15 min and was stopped by the addition of glycine to a final concentration of 0.125 M. Cells were harvested and washed twice with phosphate buffer. Cells were resuspended in buffer (150 mM NaCl, 50 mM HEPES/KOH pH7.5, 1 mM EDTA, 10% glycerol, 0.1% NP40, 0.17 mM PMSF, protease inhibitor cocktail, 100 μg/ml lysozyme) and sonicated. The solution was centrifuged and the supernatant was precleared with the addition of 50 μl of protein A beads and 50 μg of carrier DNA for 1 hour at 4° C. Precleared genomic DNA was incubated with 5 μl (1:100, vol/vol) anti-V5 monoclonal antibody (Invitrogen) or no antibody and rotated at 4° C. for 12-16 hours. Immuno-precipitation, washing and elution of immune complexes was carried out twice as previously described (Weinmann & Farnham, Methods. 26(1):37-47, 2002). Cross-links were reversed by the addition of NaCl to a final concentration of 200 mM, and RNA was removed by the addition of 10 ug of RNase A per sample followed by incubation at 65° C. for 5 hours. The samples were then precipitated at 20° C. overnight by the addition of 2.5 volumes of ethanol and then pelleted by centrifugation. The pellet was resuspended in a solution of 10 mM EDTA, 30 mM Tris (pH6.5) and 60 mg/ml proteinase K. The samples were incubated at 50° C. for 30 min and extracted with phenol-choloroform-isoamylalcohol (25:24:1, vol/vol) followed by extraction with chloroform and then precipitated. The resuspended DNA was treated with T4 DNA polymerase to create blunt-ended DNA fragments and then cloned into a pUC19 vector (Invitrogen) digested with HincII.

After reversal of the formaldehyde cross-links and purification of the DNA, the precipitated DNA fragments were cloned into vectors and sequenced to examine whether there were expected binding sequences of T9 ZFP on the intergenic region from each clone. Of 200 clones sequenced, 6 clones were identified that had perfectly or one-base mismatched binding sequences of T9 ZFP, 5′-GAA GRA HGA NGG-3′ (SEQ ID NO: 160), on their intergenic region. Since T9 ZFP was not fused with a functional domain, it was expected to function as a transcriptional repressor in E. coli (Kim and Pabo, J. Biol. Chem. 272(47):29795-800, 1997; Kang and Kim, J. Biol. Chem. 275(12):8742-8, 2000). To validate the functional relevance of T9 ZFP with thermo-tolerance phenotype in E. coli, we knocked-out each open reading frame associated with the 6 open reading frames having T9 binding sequences and examined the response of the cells to heat treatment. Strain DY330 (W3110 DlacU169 gal490 lc1857 D (cro-bioA)) was used for gene disruption by targeted homologous recombination (Yu et al., Proc Nat] Acad Sci USA. 97(11):5978-83, 2000). Linear cat (Cm^{R}) cassette with 40-bp flanking arms of target gene was amplified by PCR. Purified linear donor DNA was introduced into competent cells by electroporation and knock-out mutants were selected from growing colonies on LB plate containing chloramphenicol.

One of the gene we disrupted was the UbiX gene, which encodes 3-octaprenyl-4-hydroxybenzoate carboxy-lyase. The amino acid sequence of the UbiX gene product is shown in Table 10, below.

TABLE 10

Amino acid sequence of UbiX gene product of Escherichia coli K12;
also available in GenBank ®, GI No:1788650; Acc. No.:AAC75371.1;
encoded by nucleotides 2126-2695 in GenBank ® genomic entry
AE000320.1.

| | |
|---|---|
| MKRLIVGISGASGAIYGVRLLQVLRDVTDIETHLVMSQAARQTLSLETDFSLREVQALA | (SEQ ID NO:161) |
| DVTHDARDIAASISSGSFQTLGMVILPCSIKTLSGIVHSYTDGLLTRAADVVLKERRPLVL | |
| CVRETPLHLGHLRLMTQAAEIGAVIMPPVPAFYHRPQSLDDVINQTVNRVLDQFAITLPE | |
| DLFARWQGA | |

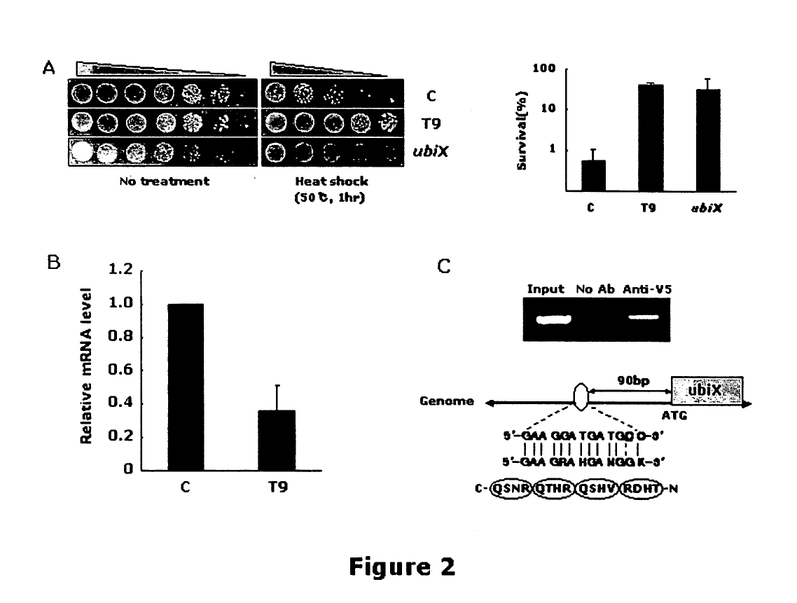

The strain in which the UbiX gene (ubiX) was knocked-out showed heat shock resistance upon heat treatment at 55° C. for 2 hrs. The effect of heat treatment on the viability of ubiX strains is shown in FIG. 2A. Plates grown from cultures of heat-shocked ubiX cells displayed far more colonies than plates grown from cultures of heat-shocked control cells.

In normal conditions, the ubiX strain grew slowly and grew small colonies on plates as compared to wild type strains. However, the ubiX strain was extremely resistant to the lethal effects of heat shock. We compared the survival rate of ubiX strain with wild type and T9 ZFP expressing strains. Suvival was compared by calculating the number of cells that survive under stress conditions divided by the number of cells that survived under normal conditions (FIG. 2A, right panel). The survival rate of ubiX and T9 strains after heat treatment was 0.42% and 0.32%, respectively, whereas the survival rate of control strains was 0.005%. To verify that the T9 ZFP was able to repress UbiX at the level of transcription, we analyzed UbiX RNA levels of E. coli transformed with T9 ZFP by RT-PCR. RNA was extracted with Trizol LS (Gibco BRL) according to the manufacturer's instructions. For the analysis of UbiX gene expression, complementary DNA synthesis was performed on RNA with UbiX-R primer (5′-CTG GAA AGA ACC GGA AGA GAT GCT G-3′) (SEQ ID NO: 162). Real-time RT PCR was performed using a Light Cycler (Corbett Research) with UbiX-F (5′-TGA AAC GAC TCA TTG TAG GCA TCA G-3′) (SEQ ID NO: 163) and UbiX-R primer sets. The RNA level of GAPDH was used as an internal control.

As expected, levels of UbiX RNA decreased more than 2 fold upon T9 ZFP expression (FIG. 2B). The UbiX gene has one-base mismatched binding site of T9 ZFP at the position of −90 bp upstream of transcriptional start codon. The in vivo binding of T9 ZFP to the target sequences of UbiX promoter was confirmed by immuno-precipitation (FIG. 2C). Combined results of in silico analysis, immuno-precipitation, gene knock-out mutation and transcriptional repression by T9 ZFP suggest that UbiX is directly regulated by T9 ZFP and that moderate repression of UbiX induces heat-shock resistance in E. coli.

UbiX functions in the biosynthesis of ubiquinone that is an essential redox component of the aerobic respiratory chains of bacteria and mitochondria (Gennis and Stewart, Escherichia coli and Salmonella: Cellular and Molecular Biology, 2^{nd} ed., p.217-261, Neidhardt et al., eds. Am Soc. Microbiol.). It has been reported that ubiquinone deficient strain, ubiCA, exhibited resistant to heat (Soballe and Poole, Microbiol. 146:787-96, 2000). It is interesting to note that knock-down expression of UbiX by ZFP, in contrast to knock-out mutation, could induce heat shock resistance without causing growth defects. This result suggests that moderate regulation of target gene expression can generate a desired phenotype in microbial engineering. ZFP library technology can be used to regulate gene expression at a range of levels.

A number of embodiments of the invention have been described. Nevertheless, it will be understood that various modifications may be made without departing from the spirit and scope of the invention. Accordingly, other embodiments are within the scope of the following claims.

What is claimed is:

1. A method of regulating expression of a gene in a prokaryotic cell, the method comprising:
providing a prokaryotic cell comprising a nucleic acid encoding an artificial polypeptide, wherein the artificial polypeptide comprises a zinc finger domain, and wherein the artificial polypeptide binds to a target DNA site in a gene;
expressing the nucleic acid encoding the artificial polypeptide in the cell under conditions in which the artificial polypeptide is produced, binds to the target DNA site, and regulates the gene.

2. The method of claim 1, wherein the artificial polypeptide comprises at least three zinc finger domains.

3. The method of claim 1, wherein the gene is an endogenous gene.

4. The method of claim 3, wherein expression of two or more endogenous genes is regulated.

5. The method of claim 4, wherein the artificial polypeptide regulates expression of a polycistronic RNA.

6. The method of claim 1, wherein expression of the gene is repressed relative to expression of the gene in the absence of the artificial protein.

7. The method of claim 1, wherein the cell is an E. coli cell.

8. The method of claim 1, wherein the regulating alters a trait of the cell relative to a reference cell.

9. The method of claim 8, wherein the trait is heat resistance or solvent resistance.

10. The method of claim 3, wherein the endogenous gene encodes a decarboxylase enzyme.

11. The method of claim 10, wherein the decarboxylase enzyme is a decarboxylase enzyme of a ubiquinone biosynthetic pathway.

12. The method of claim 11, wherein the enzyme is a ubiX gene product.

13. The method of claim 1, wherein expression of the nucleic acid encoding the artificial polypeptide is regulatable.

14. The method of claim 3, further comprising characterizing the endogenous gene.

15. The method of claim 14, wherein the characterizing comprises identifying DNA bound by the artificial polypeptide, and determining the nucleotide sequence of the endogenous gene associated with the bound DNA.

16. The method of claim 15, wherein the isolating comprises cross-linking the artificial protein to the DNA, and immunoprecipitating the artificial protein.

17. The method of claim 15, further comprising identifying a homolog of the endogenous gene in a second type of cell, and regulating the expression of the homolog.

18. The method of claim 17, wherein the second type of cell is a prokaryotic cell.

19. The method of claim 18, wherein the second type of cell is a bacterial cell.

20. A method comprising:
providing a plurality of prokaryotic cells, wherein each cell of the plurality comprises a nucleic acid encoding an artificial polypeptide, wherein the artificial polypeptide comprises a zinc finger domain, and wherein the artificial polypeptide differs among the cells of the plurality;
identifying from the plurality a cell that has a trait that is altered relative to a reference cell.

21. The method of claim 20, wherein the trait is tolerance to an organic solvent, and wherein the identifying comprises exposing cells of the plurality to the organic solvent and evaluating survival of the cells.

22. The method of claim 20, wherein the trait is heat tolerance, and wherein the evaluating comprises exposing the cells to heat.

23. The method of claim 20, further comprising isolating the nucleic acid encoding the artificial polypeptide from the identified cell.

24. The method of claim 23, further comprising sequencing the nucleic acid.

25. The method of claim 20, further comprising isolating the artificial polypeptide from the identified cell.

26. The method of claim 20, further comprising isolating the nucleic acid encoding the artificial polypeptide from the identified cell, introducing the nucleic acid into a second plurality of cells, culturing the cells of the second plurality under conditions wherein the artificial polypeptide is produced, and identifying a cell of the second plurality having a trait that is altered relative to a reference cell.

27. The method of claim 20, further comprising determining the sequence of the target DNA site of the artificial polypeptide.

28. The method of claim 20, further comprising identifying an endogenous gene bound by the artificial polypeptide.

29. The method of claim 20, further comprising analyzing the expression of one or more genes of the cell.

30. The method of claim 28, further comprising modifying expression of the endogenous gene in a second cell.

31. The method of claim 20, wherein the artificial polypeptide comprises at least three zinc finger domains.

32. The method of claim 31, wherein the zinc finger domains are yeast zinc finger domains, or variants thereof.

33. The method of claim 20, further comprising cultivating the identified cell to exploit the altered trait.

34. A prokaryotic cell comprising:
a nucleic acid encoding an artificial polypeptide, wherein the artificial polypeptide comprises a zinc finger domain, and wherein the artificial polypeptide binds to a target DNA site in a gene and regulates expression of the gene under conditions in which the nucleic acid is expressed.

35. The cell of claim 34, wherein the artificial polypeptide regulates expression of an endogenous gene.

36. The cell of claim 34, wherein the artificial polypeptide comprises at least three zinc finger domains.

37. The cell of claim 35, wherein the gene is a decarboxylase.

38. A cell selected by the method of claim 20.

39. A polypeptide comprising at least one zinc finger domain, wherein the DNA contacting residues of the zinc finger domain at positions −1, +2, +3, and +6 correspond to a motif selected from: RSHR, HSSR, ISNR, RDHT, QTHR1, VSTR, QNTQ, and CSNR, and wherein the polypeptide regulates an endogenous prokaryotic gene.

40. The polypeptide of claim 39, further comprising a second and third zinc finger domain, wherein the DNA contacting residues of the first, second, and third domains at positions −1, +2, +3, and +6 of each domain respectively correspond to the motifs RSHR,
HSSR, and ISNR.

41. The polypeptide of claim 39, further comprising a second and third zinc finger domain, wherein the DNA contacting residues of the first, second, and third domains at positions −1, +2, +3, and +6 of each domain respectively correspond to the motifs ISNR,
RDHT, and QTHR.

42. The polypeptide of claim 41, further comprising a fourth zinc finger domain, wherein the DNA contacting residues of the fourth domain at positions −1, +2, +3, and +6 of correspond to the motif VSTR.

43. The polypeptide of claim 39, further comprising a second and third zinc finger domain, wherein the DNA contacting residues of the first, second, and third domains at positions −1, +2, +3, and +6 of each domain respectively correspond to the motifs QNTQ, CSNR, and ISNR.

44. A polypeptide comprising at least one zinc finger domain, wherein the DNA contacting residues of the zinc finger domain at positions −1, +2, +3, and +6 correspond to a motif selected from: QSHV, VSNV, QSNK, RDHT, QTHR, QSSR, WSNR, VSNV RSHR, DSAR, QTHQ, RSHR, QSNR, and CSNR, and wherein the polypeptide regulates an endogenous prokaryotic gene.

45. The polypeptide of claim 44, further comprising a second, third, and fourth zinc finger domain, wherein the DNA contacting residues of the first, second, third, and fourth domains at positions −1, +2, +3, and +6 of each domain respectively correspond to the motifs QSHV, VSNV, QSNK, and QSNK.

46. The polypeptide of claim 44, further comprising a second, third, and fourth zinc finger domain, wherein the DNA contacting residues of the first, second, third, and fourth domains at positions −1, +2, +3, and +6 of each domain respectively correspond to the motifs RDHT, QSHV, QTHR, and QSSR.

47. The polypeptide of claim 44, further comprising a second, third, and fourth zinc finger domain, wherein the DNA contacting residues of the first, second, third, and fourth domains at positions −1, +2, +3, and +6 of each domain respectively correspond to the motifs WSNR, QSHV, VSNV, and QSHV.

48. The polypeptide of claim 44, further comprising a second, third, and fourth zinc finger domain, wherein the DNA contacting residues of the first, second, third, and fourth domains at positions −1, +2, +3, and +6 of each domain respectively correspond to the motifs QTHR, RSHR, QTHR, and QTHR.

49. The polypeptide of claim 44, further comprising a second, third, and fourth zinc finger domain, wherein the DNA contacting residues of the first, second, third, and fourth domains at positions −1, +2, +3, and +6 of each domain respectively correspond to the motifs DSAR, RDHT, QSHV, and QTHR.

50. The polypeptide of claim 44, further comprising a second, third, and fourth zinc finger domain, wherein the DNA contacting residues of the first, second, third, and fourth domains at positions −1, +2, +3, and +6 of each domain respectively correspond to the motifs QTHQ, RSHR, QTHR, and QTHR.

51. The polypeptide of claim 44, further comprising a second, third, and fourth zinc finger domain, wherein the DNA contacting residues of the first, second, third, and fourth domains at positions −1, +2, +3, and +6 of each domain respectively correspond to the motifs QSHV, VSNV, QSNR, and CSNR.

52. The polypeptide of claim 44, further comprising a second, third, and fourth zinc finger domain, wherein the DNA contacting residues of the first, second, third, and fourth domains at positions −1, +2, +3, and +6 of each domain respectively correspond to the motifs VSNV, QTHR, QSSR, and RDHT.

53. The polypeptide of claim 44, further comprising a second, third, and fourth zinc finger domain, wherein the DNA contacting residues of the first, second, third, and fourth domains at positions −1, +2, +3, and +6 of each domain respectively correspond to the motifs RDHT, QSHV, QTHR, and QSNR.

54. The polypeptide of claim 44, further comprising a second, third, and fourth zinc finger domain, wherein the DNA contacting residues of the first, second, third, and fourth domains at positions −1, +2, +3, and +6 of each domain respectively correspond to the motifs DSAR, RDHT, QSNK, and QTHR.

55. A nucleic acid encoding the polypeptide of claim 39.

56. A bacterial nucleic acid expression vector encoding the polypeptide of claim
